(19) United States
(12) Patent Application Publication
(10) Pub. No.: US 2005/0031578 A1
(43) Pub. Date: Feb. 10, 2005

(54) Methods of performing medical procedures which promote bone growth, compositions which promote bone growth, and methods of making such compositions

(75) Inventors: Richard Deslauriers, Woodbury, CT (US); Robert Potash, Seymour, CT (US); Aisa Sendijarevic, Troy, MI (US)

Correspondence Address: BAKER BOTTS, LLP, 910 LOUISIANA, HOUSTON, TX, 77002-4995, US

(73) Assignee: DOCTOR'S RESEARCH GROUP, INC.

(21) Appl. No.: 10/808,188

(22) Filed: Mar. 24, 2004

Related U.S. Application Data
(63) Continuation-in-part of application No. 10/395,001, filed on Mar. 24, 2003.
(60) Provisional application No. 60/366,335, filed on Mar. 22, 2002.

(51) Int. Cl. A61K 31/785; C08G 63/48; C08G 63/91

(52) U.S. Cl. 424/78.27; 525/54.2

(57) ABSTRACT

The present invention provides compositions that may be useful, inter alia, in medical procedures, methods for making such compositions, and methods of performing medical procedures using such compositions. The present invention further provides a kit that includes a first container comprising a dispensing means and a first compound; and a second container comprising a dispensing means and a second compound, wherein the first compound may include, inter alia, a naturally occurring polyol, a biocompatible synthetic polyol, and mixtures thereof, and wherein the second compound may include, inter alia, isocyanate.

### CROSS-REFERENCE TO RELATED APPLICATION

The present application is a continuation-in-part application of U.S. patent application Ser. No. 10/395,001, entitled “Methods of Performing Medical Procedures Which Promote Bone Growth, Compositions Which Promote Bone Growth, and Methods of Making Such Compositions,” which was filed on Mar. 24, 2003, and claims priority from U.S. Provisional Patent Application No. 60/366,335, entitled “Three Part Biodegradable Osteoconductive Polymer and Dispensing System,” and filed on Mar. 22, 2002, the disclosures of which are incorporated herein by reference in their entirety.

### BACKGROUND OF THE INVENTION

The present invention relates generally to compositions for use in medical procedures and methods of performing medical procedures. In particular, the present invention is directed towards compositions for use in medical procedures and methods of performing medical procedures in which the composition promotes bone growth when the composition is positioned in the vicinity of a bone of a mammal.

Human bone includes a solid mineral phase and an organic matrix which is between 90% and 95% type I collagen. The mineral phase includes, inter alia, calcium and phosphate.

The mechanical properties of bone are related to its specific type of construction and internal architecture. Although bone may be relatively light, it also may have a relatively high tensile strength. This combination of high strength coupled with relatively low weight results from, inter alia, the hollow, tubular shape of bone, the layering of bone tissue, and the internal buttressing within the organic matrix. Bone tissue may supplant membranous or fibrous tissue by a mechanism referred to as “intramembranous ossification.” Bone tissue only grows by appositional growth, e.g., the deposition of a new organic matrix on the surface of the bone by adjacent surface cells. A damaged bone repairs itself through a multiphase process. Initially, bone repair begins with an inflammatory phase, involving extensive tearing of the membrane surrounding the bone (the periosteum), rupturing of blood vessels and extensive hemorrhaging. Typically, this leads to a secondary inflammatory response of white blood cells (e.g., polymorphonuclear leukocytes, macrophages, and mononuclear cells), in an effort to prevent infection. Pluripotential mesenchymal cells from the soft tissue and within the bone marrow give rise to the osteoblast cells that synthesize bone.

Known bone replacement technologies can be divided into three transitional matrix categories. The first category relies on replacing bone with either autogenous, homologous, heterologous, or decalcified bone, followed by remodeling. As referred to herein, the term “remodeling” will be understood to mean the process by which bone is continually built and resorbed within the body. This first category may be problematic, however, because of difficulties inherent in harvesting the replacement bone, as well as the risk of transmitting blood-borne pathogens into the body of the recipient. The second category involves synthetic bone replacement, e.g., replacing bone with a bone-like mineral (e.g., crystalline hydroxyapatite or calcium pyrophosphate), followed by remodeling. Synthetic bone replacement may be problematic, however, because the replacement material may have poor tensile strength and may adhere poorly to the surrounding bone. The third category relies on replacing bone with a composition that maintains its chemical and mechanical properties without change or subsequent remodeling (e.g., titanium, stainless steel, PMMA); nevertheless, this category does not allow for the growth of new bone.

### SUMMARY OF THE INVENTION

A technical advantage of the present invention is that a composition of the present invention may be positioned in the vicinity of a bone of a mammal, e.g., in the vicinity of a damaged portion of the bone, and the composition promotes bone growth. For example, the composition can be applied to an exterior surface of the bone, dispensed in an opening formed within or through the bone, injected into the bone, positioned between two pieces of bone, or the like, without necessitating exposure of the bone, e.g., by injecting the composition through the skin using a syringe. The composition also may be molded into an implant, a screw, a plate, a prosthetic member, or the like, which may be inserted in or positioned on the bone. The composition initially may be liquid, and then may cure into a solid. For example, the composition may cure into a solid in an oxygen environment and/or a hydrophilic environment. The composition may be used to reconstruct bone, fuse bones (intravertebroinfusions), reduce or eliminate bone fractures or otherwise damaged bones, and/or regenerate missing bone, e.g., generate bone growth that fills a void within a bone. The composition also may be used to make plates, screws, prosthetic joints, or the like, and/or may act as an anchor for a suture inserted in an opening in a bone, preventing the suture from falling out of the opening after insertion. Moreover, the composition may be used as a base of a substrate in order to dilate compressed structures, e.g., vertebral disks, intramedullary nails, and in angioplasty type procedures. The newly generated bone has an internal rigid fixation similar to that of bone already present in the body, such that the generated bone is not readily damaged.

Moreover, an embodiment of the present invention facilitates ready preparation of the compositions of the present invention prior to their use in a medical procedure. According to an exemplary embodiment of the present invention, a kit that is useful in promoting bone growth comprises a first container that further comprises a dispensing means and a first compound, and a second container that further comprises a dispensing means and a second compound. The first compound and the second compound may be readily combined to form a composition of the present invention at a desired time before the performance of a medical procedure by dispensing the first compound and the second compound from their respective containers.

According to an exemplary embodiment of the present invention, a composition comprises: isocyanate; and a polyol that comprises a naturally occurring polyol and a biocompatible, synthetic polyol.

According to another exemplary embodiment of the present invention, a composition comprises a naturally occurring polyol, isocyanate, and water.

According to another exemplary embodiment of the present invention, a composition comprises a biocompatible, synthetic polyol and an isocyanate.

According to another exemplary embodiment of the present invention, a composition comprises a naturally occurring polyol and an isocyanate prepolymer that comprises a biocompatible, synthetic polyol and isocyanate.

According to another exemplary embodiment of the present invention, a composition comprises a crosslinker or chain-extender; and an isocyanate prepolymer that comprises isocyanate and a biocompatible, synthetic polyol.

According to another exemplary embodiment of the present invention, a composition comprises a polyester urethane component and at least one filler material, wherein the composition is adapted to stimulate bone growth when the composition contacts or is positioned in the vicinity of a bone of a mammal.

According to an embodiment of the present invention, a method of making a composition comprises the steps of forming a first compound by mixing a naturally occurring polyol with a biocompatible, synthetic polyol; and mixing the first compound with isocyanate.

According to an embodiment of the present invention, a method of making a composition comprises the steps of forming a first compound by mixing a naturally occurring polyol with a biocompatible, synthetic polyol; mixing the first compound with isocyanate; and permitting the first compound and the isocyanate to react to form a polyester urethane.

According to another embodiment of the present invention, a method of making a composition comprises mixing a naturally occurring polyol with isocyanate, and permitting water to be present in the composition.

According to another embodiment of the present invention, a method of making a composition comprises mixing a naturally occurring polyol with isocyanate, permitting the naturally occurring polyol and the isocyanate to react to form a polyester urethane, and permitting water to be present in the composition.

According to another embodiment of the present invention, a method of making a composition comprises the step of mixing a biocompatible, synthetic polyol with an isocyanate.

According to another embodiment of the present invention, a method of making a composition comprises the steps of mixing a biocompatible, synthetic polyol with an isocyanate; and permitting the biocompatible, synthetic polyol and the isocyanate to react to form a polyester urethane.

According to yet another embodiment of the present invention, a method of making a composition comprises the steps of forming an isocyanate prepolymer by mixing a biocompatible, synthetic polyol with isocyanate; and mixing the isocyanate prepolymer with a naturally occurring polyol.

According to yet another embodiment of the present invention, a method of making a composition comprises the steps of forming an isocyanate prepolymer by mixing a biocompatible, synthetic polyol with isocyanate; mixing the isocyanate prepolymer with a naturally occurring polyol; and permitting the isocyanate prepolymer and the naturally occurring polyol to react to form a polyester urethane.

According to still yet another embodiment of the present invention, a method of making a composition comprises the step of forming an isocyanate prepolymer by mixing a biocompatible, synthetic polyol with isocyanate; and mixing the isocyanate prepolymer with a crosslinker or chain-extender.

According to still yet another embodiment of the present invention, a method of making a composition comprises the step of forming an isocyanate prepolymer by mixing a biocompatible, synthetic polyol with isocyanate; mixing the isocyanate prepolymer with a crosslinker or chain-extender; and permitting the isocyanate prepolymer and the crosslinker or chain-extender to react to form a polyester urethane.

According to an embodiment of the present invention, a method of performing a medical procedure comprises at least one step selected from the group consisting of: applying a particular composition to at least one portion of a bone of a mammal, positioning the particular composition in the vicinity of the bone, dispensing the particular composition into an opening formed within or through at least one portion of the bone, and positioning the particular composition between a first bone portion of the mammal and a second bone portion of the mammal for fusing the first bone portion to the second bone portion, wherein the particular composition stimulates bone growth and is a product of a process that comprises the steps of: forming a first compound by mixing a naturally occurring polyol with a biocompatible, synthetic polyol; mixing the first compound with isocyanate; and permitting the first compound and the isocyanate to react to form a polyester urethane.

According to an embodiment of the present invention, a method of performing a medical procedure comprises at least one step selected from the group consisting of: applying a particular composition to at least one portion of a bone of a mammal, positioning the particular composition in the vicinity of the bone, dispensing the particular composition into an opening formed within or through at least one portion of the bone, and positioning the particular composition between a first bone portion of the mammal and a second bone portion of the mammal for fusing the first bone portion to the second bone portion, wherein the particular composition stimulates bone growth and is a product of a process that comprises the steps of: mixing a naturally occurring polyol with isocyanate; permitting the naturally occurring polyol and the isocyanate to react to form a polyester urethane; and permitting water to be present in the composition.

According to an embodiment of the present invention, a method of performing a medical procedure comprises at least one step selected from the group consisting of: applying a particular composition to at least one portion of a bone of a mammal, positioning the particular composition in the vicinity of the bone, dispensing the particular composition into an opening formed within or through at least one portion of the bone, and positioning the particular composition between a first bone portion of the mammal and a second bone portion of the mammal for fusing the first bone portion to the second bone portion, wherein the particular composition stimulates bone growth and is a product of a process that comprises the steps of mixing a biocompatible, synthetic polyol with an isocyanate; and permitting the biocompatible, synthetic polyol and the isocyanate to react to form a polyester urethane.

According to an embodiment of the present invention, a method of performing a medical procedure comprises at least one step selected from the group consisting of: applying a particular composition to at least one portion of a bone of a mammal, positioning the particular composition in the vicinity of the bone, dispensing the particular composition into an opening formed within or through at least one portion of the bone, and positioning the particular composition between a first bone portion of the mammal and a second bone portion of the mammal for fusing the first bone portion to the second bone portion, wherein the particular composition stimulates bone growth and is a product of a process that comprises the steps of: forming an isocyanate prepolymer by mixing a biocompatible, synthetic polyol with isocyanate; mixing the isocyanate prepolymer with a naturally occurring polyol; and permitting the isocyanate prepolymer and the naturally occurring polyol to react to form a polyester urethane.

According to an embodiment of the present invention, a method of performing a medical procedure comprises at least one step selected from the group consisting of: applying a particular composition to at least one portion of a bone of a mammal, positioning the particular composition in the vicinity of the bone, dispensing the particular composition into an opening formed within or through at least one portion of the bone, and positioning the particular composition between a first bone portion of the mammal and a second bone portion of the mammal for fusing the first bone portion to the second bone portion, wherein the particular composition stimulates bone growth and is a product of a process that comprises the steps of: forming an isocyanate prepolymer by mixing isocyanate with a biocompatible, synthetic polyol; mixing the isocyanate prepolymer with a chain-extender or crosslinker; and permitting the chain-extender or crosslinker and the isocyanate prepolymer to react to form a polyester urethane.

According to another embodiment of the present invention, a method of performing a medical procedure comprises the steps of: forming a mold; dispensing a liquid, particular composition into the mold, wherein the particular composition solidifies within the mold; removing the solidified, particular composition from the mold; and positioning the solidified, particular composition on a bone of a mammal or within an opening formed through or in the bone; wherein the positioned, particular composition stimulates bone growth; and the particular composition is a product of a process that comprises the steps of: forming a first compound by mixing a naturally occurring polyol with a biocompatible, synthetic polyol; and mixing the first compound with isocyanate.

According to another embodiment of the present invention, a method of performing a medical procedure comprises the steps of: forming a mold; dispensing a liquid, particular composition into the mold, wherein the particular composition solidifies within the mold; removing the solidified, particular composition from the mold; and positioning the solidified, particular composition on a bone of a mammal or within an opening formed through or in the bone; wherein the positioned, particular composition stimulates bone growth; and the particular composition is a product of a process that comprises the steps of: mixing a naturally occurring polyol with isocyanate; and permitting water to be present in the composition.

According to another embodiment of the present invention, a method of performing a medical procedure comprises the steps of: forming a mold; dispensing a liquid, particular composition into the mold, wherein the particular composition solidifies within the mold; removing the solidified, particular composition from the mold; and positioning the solidified, particular composition on a bone of a mammal or within an opening formed through or in the bone; wherein the positioned, particular composition stimulates bone growth; and the particular composition is a product of a process that comprises the step of mixing a biocompatible, synthetic polyol with an isocyanate.

According to another embodiment of the present invention, a method of performing a medical procedure comprises the steps of: forming a mold; dispensing a liquid, particular composition into the mold, wherein the particular composition solidifies within the mold; removing the solidified, particular composition from the mold; and positioning the solidified, particular composition on a bone of a mammal or within an opening formed through or in the bone; wherein the positioned, particular composition stimulates bone growth; and the particular composition is a product of a process that comprises the steps of: forming an isocyanate prepolymer by mixing a biocompatible, synthetic polyol with isocyanate; and mixing the isocyanate prepolymer with a naturally occurring polyol.

According to another embodiment of the present invention, a method of performing a medical procedure comprises the steps of: forming a mold; dispensing a liquid, particular composition into the mold, wherein the particular composition solidifies within the mold; removing the solidified, particular composition from the mold; and positioning the solidified, particular composition on a bone of a mammal or within an opening formed through or in the bone; wherein the positioned, particular composition stimulates bone growth; and the particular composition is a product of a process that comprises the steps of: forming an isocyanate prepolymer by mixing isocyanate with a biocompatible, synthetic polyol; and mixing the isocyanate prepolymer with a chain-extender or crosslinker.

Other objects, features, and advantages will be apparent to persons of ordinary skill in the art in view of the following detailed description of the invention and the accompanying drawings.

### BRIEF DESCRIPTION OF THE DRAWINGS

For a more complete understanding of the present invention, needs satisfied thereby, and objects, features, and advantages thereof, reference now is made to the following descriptions taken in connection with the accompanying drawings.

FIG. 1 is a top level flow-chart depicting a method for making a composition according to an exemplary embodiment of the present invention.

FIG. 2 is a top level flow-chart depicting a method for making a composition according to another exemplary embodiment of the present invention.

FIG. 3 is a top level flow-chart depicting a method for making a composition according to yet another exemplary embodiment of the present invention.

FIG. 4 is a top level flow-chart depicting a method for making a composition according to still another exemplary embodiment of the present invention.

FIG. 5 is a top level flow-chart depicting a method for making a composition according to still yet another exemplary embodiment of the present invention.

FIG. 6 is a top level flow-chart depicting a method for making a composition according to still yet another exemplary embodiment of the present invention.

FIG. 7 is a top level flow-chart depicting a method for making a composition according to still yet another exemplary embodiment of the present invention.

FIG. 8 is a top level flow-chart depicting a method for making a composition according to still yet another exemplary embodiment of the present invention.

FIG. 9 is a top level flow-chart depicting a method for making a composition according to still yet another exemplary embodiment of the present invention.

FIG. 10 is a top level flow-chart depicting a method for making a composition according to still yet another exemplary embodiment of the present invention.

FIG. 11 is a top level flow-chart depicting a method for making a composition according to still yet another exemplary embodiment of the present invention.

FIG. 12 is a top level flow-chart depicting a method for making a composition according to still yet another exemplary embodiment of the present invention.

FIG. 13 is a top level flow-chart depicting a method for making a composition according to still yet another exemplary embodiment of the present invention.

FIG. 14 is a top level flow-chart depicting a method for making a composition according to still yet another exemplary embodiment of the present invention.

FIG. 15 is a top level flow-chart depicting a method for making a composition according to still yet another exemplary embodiment of the present invention.

FIG. 16 is a top level flow-chart depicting a first method for performing a medical procedure according to an exemplary embodiment of the present invention.

FIG. 17 is a top level flow-chart depicting a second method for performing a medical procedure according to another exemplary embodiment of the present invention.

FIG. 18 is a top level flow-chart depicting a third method for performing a medical procedure according to yet another exemplary embodiment of the present invention.

FIG. 19 is a top level flow-chart depicting a fourth method for performing a medical procedure according to still another exemplary embodiment of the present invention.

FIG. 20 is a top level flow-chart depicting a fifth method for performing a medical procedure according to still yet another exemplary embodiment of the present invention.

FIG. 21 is a top level flow-chart depicting a sixth method for performing a medical procedure according to a further exemplary embodiment of the present invention.

FIG. 22 is a top level flow-chart depicting a seventh method for performing a medical procedure according to yet a further exemplary embodiment of the present invention.

While the present invention is susceptible to various modifications and alternative forms, specific exemplary embodiments thereof have been shown in the drawings and are herein described. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

### DETAILED DESCRIPTION OF PREFERRED EMBODIMENTS

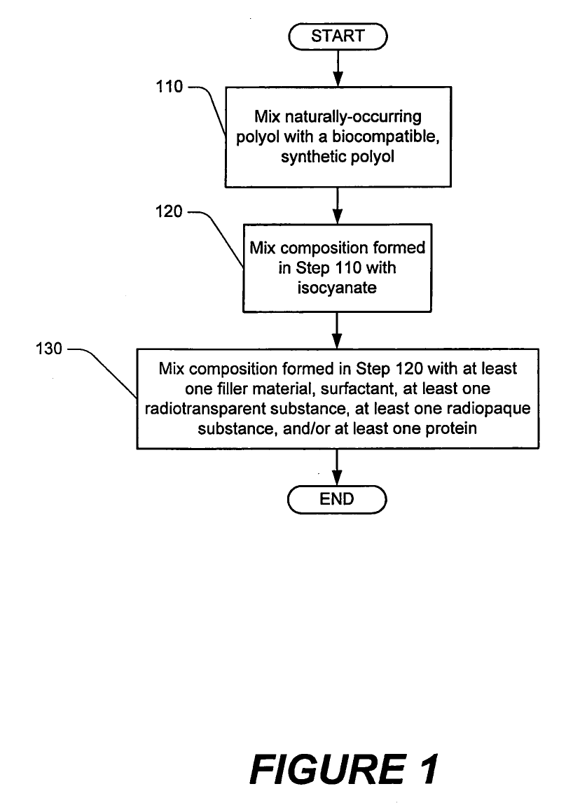

Exemplary embodiments of the present invention and their advantages may be understood by referring to FIGS. 1-22, like numerals being used for like corresponding parts in the various drawings.

According to an exemplary embodiment of the present invention, a composition of the present invention, such as a composition for use in a medical procedure, generally comprises an isocyanate and a polyol and/or a polyamine. Optionally, the compositions of the present invention also may comprise at least one filler material, at least one catalyst, and other additives (e.g., surfactants, proteins, etc.).

Another example of a composition of the present invention is a composition that comprises a biocompatible polyester urethane component. Generally, the compositions of the present invention that comprise a biocompatible polyester urethane component are those that have been prepared by combining an isocyanate with one or more polyols and/or polyamines, along with optional additives, and permitting them to react to form a composition that comprises a biocompatible polyester urethane component. The biocompatible polyester urethane component may be present in certain exemplary embodiments of the compositions of the present invention in an amount in the range of from about 40% to about 90% by weight. In certain exemplary embodiments of the compositions of the present invention, the biocompatible polyester urethane component is present in the composition in an amount in the range of from about 50% to about 70% by weight. Optionally, the compositions comprising a biocompatible polyester urethane component further may comprise other additives (e.g., at least one filler material, at least one catalyst, surfactants, proteins, etc.), as will be described.

When placed within the body of a mammal according to the methods of the present invention, the compositions of the present invention that comprise a biocompatible polyester urethane component may provide significant advantages over conventional biological materials. For example, other conventional biological materials (e.g., those comprising polylactic acid) may degrade over a particular period of time, irrespective of whether new bone has formed in the vicinity of the material. Such degradations may limit the stability of surrounding structures within the body of the mammal. In contrast to these conventional biological materials, the compositions of the present invention are degraded through a cyclic AMP regulated lipase hydrolysis reaction process that generally maintains a 1-to-1 conversion (e.g., the compositions of the present invention generally are replaced or converted to bone while maintaining regional structural stability). Additionally, conventional biological materials with osteoconductive properties often have an open cellular matrix to support osteoclasts and osteoblasts in growth. While certain exemplary embodiments of the compositions of the present invention may be considered to have a closed cellular matrix, the compositions of the present invention support osteoclasts in growth through local degradations that create micro-open-cellular matrices. Such micro-open-cellular matrices support osteoclast and osteoblast activity, e.g., the osteoclast metabolic activities create an open-cell matrix.

The compositions of the present invention that comprise a biocompatible polyester urethane component generally reside in a moldable state at room temperature for a desired time. Certain exemplary embodiments of the compositions of the present invention that comprise a biocompatible polyester urethane component may reside in a moldable state at room temperature for up to about 20 minutes after their formulation; certain other exemplary embodiments may reside in a moldable state at room temperature for a longer or shorter period of time. Absent supplemental heating or cooling, the compositions of the present invention that comprise a biocompatible polyester urethane component generally may solidify at room temperature at a time in the range of from about 20 minutes to about 30 minutes after their formulation. Where an operator desires to solidify the compositions of the present invention, the time required for solidification may be extended by cooling the composition, or may be reduced by heating the composition. Within about 48 hours after their formulation, certain exemplary embodiments of the compositions of the present invention that comprise a biocompatible polyester urethane component may attain a final, cured state; certain other exemplary embodiments of the compositions of the present invention that comprise a biocompatible polyester urethane component may attain a final, cured state at an earlier time. Certain exemplary embodiments of the compositions of the present invention that comprise a biocompatible polyester urethane component may have an average porosity in the range of from about 5 to about 500 microns; certain other exemplary embodiments may have an average porosity that is less, or that is greater. Certain exemplary embodiments of the compositions of the present invention that comprise a biocompatible polyester urethane component may have an average porosity in the range of from about 5 to about 100 microns. Certain exemplary embodiments of the compositions of the present invention that comprise a biocompatible polyester urethane component are resistant to thermal degradation up to temperatures of about 300 F. Certain exemplary embodiments may be capable of withstanding traditional autoclave sterilization cycles. For example, certain exemplary embodiments of the compositions of the present invention that comprise a biocompatible polyester urethane component repeatedly may be sterilized in an autoclave without substantially affecting their mechanical properties. Certain exemplary embodiments of the compositions of the present invention that comprise a biocompatible polyester urethane component demonstrate a compressive strength of at least about 50 MPa, a tensile strength of at least about 40 MPa, and/or a Young's Modulus of Elasticity of at least about 1,500 MPa. Certain other exemplary embodiments may demonstrate greater or lesser compressive strength, tensile strength, and/or Young's Modulus.

The compositions of the present invention are biocompatible, and are adapted to stimulate bone growth when positioned in contact with, or in the vicinity of, a bone of a mammal. Certain exemplary embodiments of the compositions of the present invention may be adhesive and cohesive, and certain exemplary embodiments may be bacterial static and bactericidal. Certain exemplary embodiments of the compositions of the present invention also may be osteoinductive or osteoconductive. Certain exemplary embodiments of the compositions of the present invention may be suitable for use as a USP Class VI medical adhesive.

The Isocyanate Component

A broad variety of isocyanates may be suitable for use in the compositions and methods of the present invention. In certain exemplary embodiments of the present invention, the isocyanate may be, e.g., an aromatic isocyanate, an aliphatic isocyanate, a cycloaliphatic isocyanate, or the like. An example of a suitable aromatic isocyanate is diphenylmethane isocyanate, also known as “MDI.” Commercially available examples of diphenylmethane isocyanate include, but are not limited to, mixtures of 2,4-diphenylmethane isocyanate and 4,4-diphenylmethane isocyanate isomers, such as those that are commercially available from Dow Chemical Company under the tradename ISONATE 50 OP, and those that are commercially available from Huntsman under the tradename RUBINATE 9433; these mixtures of 2,4- and 4,4-diphenylmethane isocyanate isomers generally will be liquids at room temperatures. Diphenylmethane isocyanate is also commercially available, inter alia, in its pure 4,4-diphenylmethane isocyanate form from Bayer AG under the tradename MONDUR M, and from Huntsman Corporation under the tradename RUBINATE 44; these compounds generally will be solids at room temperature. Other examples of suitable aromatic isocyanates include, but are not limited to, polymeric isocyanates, such as those that are commercially available from Dow Chemical Company under the tradenames ISONATE 143L, ISONATE PAPI 901, ISONATE PAPI 27, and the like. Examples of suitable cycloaliphatic isocyanates include, but are not limited to, isophorone diisocyanate and dicyclohexylmethane diisocyanate. Isophorone diisocyanate is commercially available from Bayer Corporation under the trade name DESMODUR I. Dicyclohexyl methane diisocyanate is commercially available from Bayer Corporation under the trade name DESMODUR W. An example of a suitable aliphatic isocyanate is 1,6 hexylmethylene diisocyanate.

In certain exemplary embodiments of the present invention, the isocyanate may be chosen so that the compositions of the present invention that comprise the isocyanate will be liquids at room temperature and will have desired flexural properties. For example, an exemplary embodiment of a composition of the present invention that was prepared from an isocyanate prepolymer that comprised ISONATE PAPI 901 demonstrated flexural strength of 64.9 MPa at 5.5% strain (no yield), while another exemplary embodiment of a composition of the present invention that was prepared from an isocyanate prepolymer that comprised ISONATE 500P exhibited flexural strength of 57.4 MPa at 7.0% strain (yield). Furthermore, the quantity of isocyanate that may be included in the compositions of the present invention will also depend on factors including, inter alia, the desired flexural properties of the composition. In certain exemplary embodiments of the present invention where the composition comprises a isocyanate prepolymer that comprises an isocyanate, the isocyanate is generally present in the isocyanate prepolymer in an amount in the range of from about 30% to about 80% by weight of the isocyanate prepolymer. In certain exemplary embodiments, the isocyanate may be present in the isocyanate prepolymer in an amount in the range of from about 30% to about 70% by weight of the isocyanate prepolymer. One of ordinary skill in the art, with the benefit of this disclosure, will be able to identify a suitable amount of isocyanate to include in the compositions of the present invention for a particular application.

The Polyol/Polyamine Component

The compositions of the present invention further may comprise a polyol and/or a polyamine. A broad variety of polyols may be suitable for use in the compositions and methods of the present invention, including, but not limited to, naturally occurring polyols and biocompatible, synthetic polyols, and mixtures thereof. The polyols used in the present invention generally comprise at least one ester group. In certain exemplary embodiments, a polyol used in the present invention may comprise in the range of from about 2 to 3 ester groups. In certain exemplary embodiments, a polyol used in the present invention may comprise in the range of from about 5 to 10 ester groups.

As referred to herein, the term “naturally occurring polyols” will be understood to include, inter alia, naturally occurring polyols as well as polyols that are derived from various vegetable oils. Generally, the naturally occurring polyols that are suitable for use in the present invention are those that have at least one hydroxyl group. In certain exemplary embodiments, the naturally occurring polyols have two or more hydroxyl groups. Examples of naturally occurring polyols include, but are not limited to, castor oil, safflower oil, lesquerella oil, the polyols that may be obtained by chemical modification of naturally occurring vegetable oils (e.g., castor oil, olive oil, sesame oil, corn oil), naturally occurring oils that have been trans-esterified (e.g., a modified castor oil polyol that has been prepared by the transesterification reaction of natural castor oil with suitable crosslinkers (e.g., glycerol, trimethylolpropane, and the like)), naturally occurring oils that have been hydrogenated, and the like. Another example of a suitable naturally occurring polyol is a difunctional castor-oil-based polyol that is commercially available from CasChem, Inc., under the tradename CASPOL® 5001.

As referred to herein, the term “biocompatible, synthetic polyols” will be understood to include, inter alia, biocompatible synthetic polyols that are derived from crude oil. Examples of suitable biocompatible, synthetic polyols include, but are not limited to, polycaprolactone polyols, polyester polyols, polyadipate polyols (e.g., poly(hexane-adipate) diol, poly(butane-adipate) diol, poly(ethylene/propylene-adipate) diol, poly(hexane/adipate/isophthalate diol)), polyols that have been derived from a synthetic acid (e.g., isophthalic acid, maleic acid), and the like. In certain exemplary embodiments, the biocompatible, synthetic polyol may be biodegradable. An example of a suitable polycaprolactone polyol is a polycaprolactone diol that is commercially available from Dow Chemical under the trade name TONE 32 B8.

In certain exemplary embodiments of the present invention, an isocyanate prepolymer may be reacted with a polyamine to form a polyurethane according to a method of the present invention. In certain exemplary embodiments, the polyamine may be a product of a chemical transformation of a naturally occurring polyol. In certain exemplary embodiments, the polyamine may be a secondary di-amine. The choice of a particular polyol or polyamine for use in accordance with the present invention may depend on factors including, inter alia, the desired flexural properties of the compositions of the present invention that are produced from the particular polyol or polyamine. The use of a relatively short-chain polyol or polyamine will tend to impart less flexibility to the composition of the present invention than will the use of a relatively long-chain polyol or polyamine. One of ordinary skill in the art, with the benefit of this disclosure, will be able to identify a suitable polyol or polyamine for a particular application.

In certain exemplary embodiments of the present invention, the polyol or polyamine is generally present in the isocyanate prepolymer in an amount in the range of from about 10% to about 50% by weight of the isocyanate prepolymer. In certain exemplary embodiments, the polyol or polyamine may be present in the isocyanate prepolymer in an amount in the range of from about 20% to about 35% by weight of the isocyanate prepolymer. One of ordinary skill in the art, with the benefit of this disclosure, will also be able to identify a suitable amount of polyol or polyamine to include in the compositions of the present invention for a particular application.

The Chain-Extender/Crosslinker Component

Examples of suitable crosslinkers include, but are not limited to, trimethylolpropane, glycerine, a trifunctional castor-oil-based polyol that is commercially available from CasChem, Inc., under the tradename CASPO® 1962, a quadrifunctional castor-oil-based polyol that is commercially available from CasChem, Inc., under the tradename CASPOL® 5004, and the like.

Examples of suitable chain-extenders include, but are not limited to, 1,4-butanediol, 1,6-hexanediol, diethylene glycol, and the like.

In certain exemplary embodiments wherein a composition of the present invention comprises a chain-extender or crosslinker, the chain-extender or crosslinker may be present in the composition in an amount in the range of about 50% to about 80% by weight of the isocyanate prepolymer. In certain exemplary embodiments, the chain-extender or crosslinker may be present in the composition in an amount in the range of about 60% to about 70% by weight of the isocyanate prepolymer.

Water

Optionally, the compositions of the present invention also may comprise water. The water may be added to the compositions of the present invention in a variety of ways. For example, formulating the compositions of the present invention in an atmosphere that contains moisture may cause water to become incorporated into the compositions. As another example, an operator may add a desired amount of water by injecting it into the compositions of the present invention during their formulation. Because water is known to react with isocyanate to produce carbon dioxide, the presence of water in certain exemplary compositions of the present invention that also comprise an isocyanate may generate a sufficient amount of carbon dioxide to impart a degree of porosity to the compositions of the present invention. In certain exemplary embodiments of the present invention, water is present in the composition in an amount sufficient to provide a desired porosity. In certain exemplary embodiments, water may be present in the compositions of the present invention in an amount in the range of from about 0.1% to about 1% by weight of the composition. One of ordinary skill in the art, with the benefit of this disclosure, will be able to identify an appropriate amount of water to include in the compositions of the present invention for a particular application.

The Optional Filler Material Component

A broad variety of filler materials may be suitable for use in the compositions of the present invention, including, but not limited to, calcium carbonate, bone (e.g., demineralized bone, allograft bone, and/or autogenous bone), calcium phosphate, calcium pyrophosphate, hydroxyapatite, poly methyl methacrylate, glass-ionomer, calcium sulfate, tricalcium phosphate (e.g., beta tricalcium phosphate), or any combination thereof, or the like. In certain exemplary embodiments, the filler material may be chosen so as to impart a desired degree of porosity in the compositions of the present invention. Generally, the greater the adhesion between the filler material and other components in the composition, the lower the composition's porosity; and vice versa. Generally, the filler material will be present in the compositions of the present invention in an amount sufficient to modify the composition's mechanical properties (e.g., Young's Modulus of Elasticity, flexural strength, and the like). In certain exemplary embodiments, the filler material is present in the compositions of the present invention in an amount in the range of from about 0.01% to about 55% by weight. In certain exemplary embodiments, the filler material may be present in the compositions of the present invention in an amount in the range of from about 25% to about 35% by weight. In certain exemplary embodiments, the filler material comprises calcium carbonate. In certain of these embodiments, the filler material comprises calcium carbonate in an amount sufficient to provide free calcium to a body of a mammal and enhance osteoconductivity. In certain exemplary embodiments, the filler material comprises at least about 98% pure calcium carbonate by weight of the filler material. In certain exemplary embodiments, the calcium carbonate is implantable grade calcium carbonate. In certain exemplary embodiments, the calcium carbonate has a particle size distribution that is capable of enhancing resorption of calcium within the body of a mammal. In certain exemplary embodiments, the particle size distribution further enhances bone remodeling. An example of a suitable filler material is poly ether ether ketone (often referred to as “PEEK”). Another example of a suitable filler material is commercially available from Cortek, Inc., of Dedham, Mass., under the trade name “Replace™.”

The Optional Catalyst Component

Optionally, certain exemplary embodiments of the compositions of the present invention further may comprise at least one catalyst. In certain exemplary embodiments of the present invention where a catalyst is used, the catalyst may be used by adding the catalyst to a polyol that may be mixed into the compositions of the present invention. The inclusion of the catalyst in the compositions and methods of the present invention may permit an operator to control, inter alia, certain polymerization reactions that occur during the formulation of the compositions of the present invention (e.g., a polymerization reaction between a polyol and a isocyanate prepolymer that comprises an isocyanate). In certain exemplary embodiments of the present invention, at least one catalyst is present in the compositions of the present invention in an amount sufficient to ensure that such polymerization reactions have proceeded to completion for the compositions of the present invention are placed within the body of a mammal. This may ensure, inter alia, that the isocyanate that may be present within the compositions of the present invention at the time of their placement within the body of a mammal is not free to react while within the body.

A broad variety of catalysts may be used, including, but not limited to, a tertiary amine, and organometallic compounds such as, for example, stannous octoate, and dibutyl tin dilaurate. In certain exemplary embodiments wherein the catalyst is an organometallic catalyst, the presence of the organometallic catalyst in the compositions of the present invention will not adversely impact the radiotransparency or radiopacity of the composition. An example of a suitable tertiary amine is commercially available from Air Products, Inc., under the trade name DABCO 33LV. An example of a suitable source of dibutyl tin dilaurate is commercially available from Air Products, Inc., under the trade name DABCO T12. A tertiary amine may be preferred in, inter alia, certain exemplary embodiments of the present invention wherein a catalyst is to be used during preparation of a composition of the present invention that may be placed within a body of a mammal while in liquid form.

In certain exemplary embodiments of the compositions of the present invention that comprise a catalyst, the catalyst will remain in the composition after its formulation and curing, e.g., as a monomer that is present in the matrix of the cured composition. In certain exemplary embodiments of the present invention where a catalyst is added to a polyol that may be mixed into the compositions of the present invention, the catalyst may be present in the polyol in an amount in the range of from about 0.05% to about 0.5% by weight of the polyol. In certain exemplary embodiments, the catalyst may be present in the polyol in an amount in the range of from about 0.15% to about 0.4% by weight of the polyol. One of ordinary skill in the art, with the benefit of this disclosure, will be able to identify a suitable at least one catalyst, and a suitable amount for inclusion in the compositions of the present invention, for a particular application.

The Optional Surfactant Component

Optionally, certain exemplary embodiments of the compositions of the present invention further may comprise at least one surfactant. The inclusion of the at least one surfactant in the compositions and methods of the present invention may, inter alia, impart a desired degree of porosity to the composition, and may permit an operator to control, inter alia, the size and/or the shape of pores within the composition. A broad variety of surfactants may be suitable for inclusion in the compositions of the present invention. Commercially available examples of suitable surfactants include, but are not limited to, DABCO DC 193 and DABCO DC 5241, both of which are commercially available from Air Products, Inc., as well as copolymerizable surfactants with phosphate ester functionality that are available under the tradenames “MAXEMUL 6106” and “MAXEMUL 6112” from Uniqema, and silicone surfactants that are commercially available from Struktol Corporation. One of ordinary skill in the art, with the benefit of this disclosure, will be able to identify an appropriate amount of surfactant to include in the compositions of the present invention for a particular application.

The Optional Radiotransparent/Radiopaque Component

Optionally, the compositions of the present invention also may comprise at least one radiotransparent substance or at least one radiopaque substance. The inclusion of such radiotransparent or radiopaque substances in the compositions of the present invention may be useful, inter alia, when the composition has been placed in contact with, or in the vicinity of, a bone of a mammal, and a physician subsequently seeks to determine the condition or location of the composition or the bone through the use of, inter alia, X-ray photographs. When an exemplary embodiment of the compositions of the present invention includes a radiotransparent substance (e.g., air, nitrogen gas, carbon dioxide, oxygen gas, or the like), the attenuation of the composition in the X-ray decreases. Consequently, a physician more readily may visualize the extent to which the underlying damaged bone has been repaired by the bone growth facilitated by treatment with the compositions of the present invention. Similarly, when an exemplary embodiment of the compositions of the present invention includes a radiopaque substance (e.g., insoluble zirconium oxide, a radioactive tracer, a Barium Sulfate contrast media, a gadolinium contrast media, a water-soluble Iodinated contrast media, an oily Iodinated contrast media, an implantable metal (e.g., a chip, flake, or the like comprising a metal such as titanium, cobalt, or chromium), or the like), the attenuation of the composition in the X-ray increases. Consequently, the physician more readily may visualize the adequacy of the coverage of the compositions of the present invention on the damaged bone. Moreover, the at least one radiotransparent substance and/or the at least one radiopaque substance may be non-reactive substances, such that they may be included within the compositions of the present invention at any time during the process of manufacturing the compositions. Generally, the at least one radiotransparent substance and/or the at least one radiopaque substance may be present in the compositions of the present invention in an amount in the range of from about 5% to about 30% by weight of the composition. In certain exemplary embodiments, the at least one radiotransparent substance and/or the at least one radiopaque substance may be present in the compositions of the present invention in an amount in the range of from about 10% to about 20% by weight of the composition. Examples of commercially available radiopaque substances include “LIPIODOL,” “HYPAQUE,” and “OMNIPAQUE.”

Other Optional Additives

Optionally, the compositions of the present invention further may comprise at least one protein. The at least one protein may be used to control the rate of bone regrowth, e.g., the type of protein may be selected, such that the protein increases or decreases the rate of bone growth relative to when the at least one protein is not present in the composition. For example, when a physician wishes to closely monitor the bone growth produced by treatment with the compositions of the present invention, the physician may opt to include within the composition at least one protein that tends to decrease the rate of bone growth relative to when the at least one protein is not present in the composition. Moreover, the at least one protein may be non-reactive, such that the at least one protein may be included in the compositions of the present invention at any time during the manufacture of the composition. Examples of suitable proteins include, but are not limited to, OPI (commercially available from Stryker Homedica), or any recombinant bone morphogenic protein. Further, at least one steroid-based intracellular messenger may be included in the compositions of the present invention, inter alia, to modulate the rate of bone growth.

Methods of Use

FIGS. 1 through 15 set forth exemplary embodiments of methods of making certain exemplary embodiments of the compositions of the present invention. Referring now to FIG. 1, a first compound is formed in step 110 by mixing a naturally occurring polyol with a biocompatible, synthetic polyol. In step 120, the first compound formed in step 110 may be mixed with an isocyanate. In certain exemplary embodiments, the isocyanate and the first compound both may be liquids at room temperature. In an exemplary embodiment, in step 130, the mixture formed in step 120 further may be mixed with at least one filler material, a surfactant, at least one radio transparent substance, at least one radiopaque substance, and/or at least one protein. The process then proceeds to end. As an alternative, the additives added in step 130 may be added earlier, e.g., in a step (not shown) between step 110 and step 120, as opposed to after step 120; in such alternative embodiments, the process would proceed to end after step 120, the optional additives already having been added in a step (not shown) between step 110 and step 120.

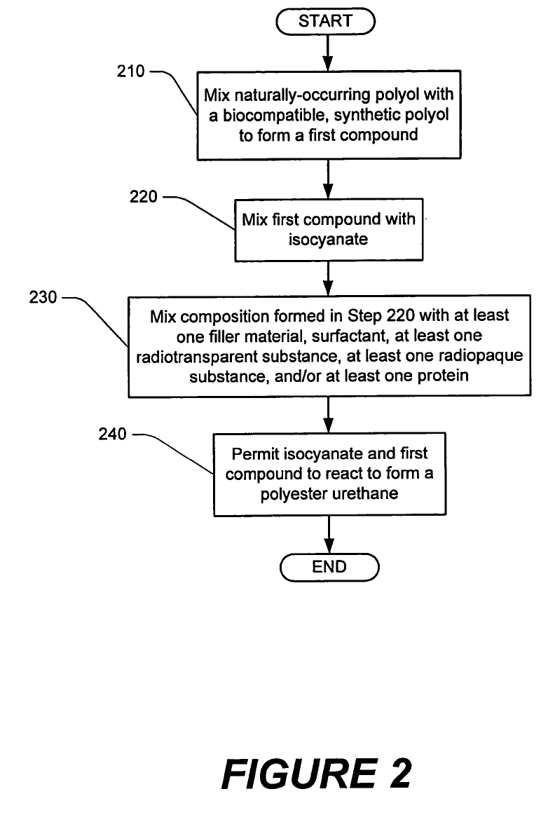

FIG. 2 illustrates an exemplary method for producing a composition of the present invention that comprises a polyester urethane. A first compound is formed in step 210 by mixing a naturally occurring polyol with a biocompatible, synthetic polyol. In step 220, the first compound formed in step 210 may be mixed with an isocyanate. In certain exemplary embodiments, the isocyanate and the first compound both may be liquids at room temperature. In an exemplary embodiment, in step 230, the mixture formed in step 220 further may be mixed with at least one filler material, a surfactant, at least one radio transparent substance, at least one radiopaque substance, and/or at least one protein. In step 240, the first compound and the isocyanate are permitted to react to form a polyester urethane. The process then proceeds to end. As an alternative, the additives added in step 230 may be added earlier, e.g., in a step (not shown) between step 210 and step 220, as opposed to after step 220; in such alternative embodiments, the process would proceed to step 240 after step 220, the optional additives already having been added in a step (not shown) between step 210 and step 220.

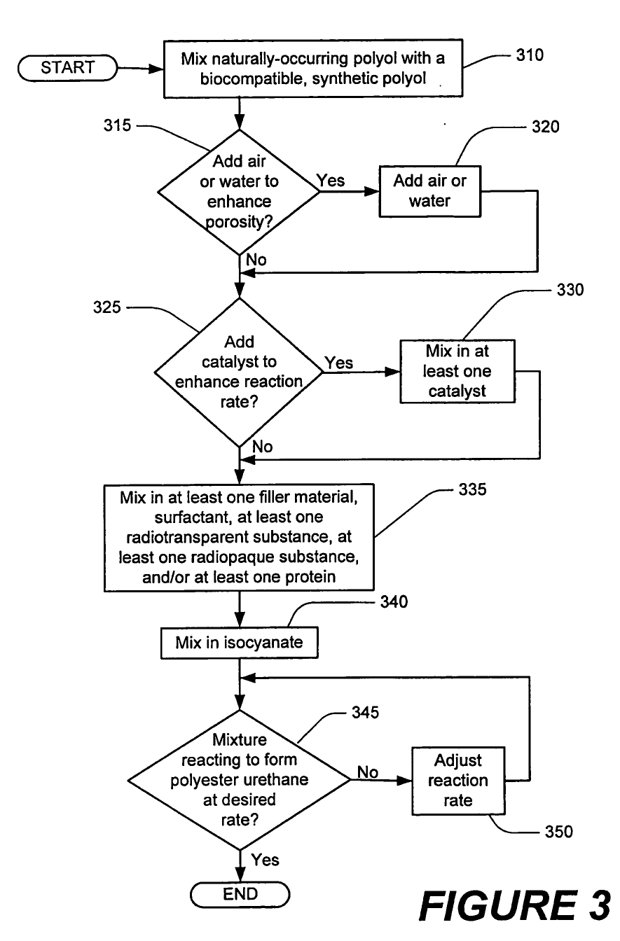

FIG. 3 illustrates an exemplary method for producing a composition of the present invention that comprises a polyester urethane. A first compound is formed in step 310 by mixing a naturally occurring polyol with a biocompatible, synthetic polyol. In step 315, the operator determines whether or not to add air or water to the mixture in an amount that may, inter alia, enhance the porosity of the final composition. If the operator elects not to add the air or water, the process proceeds to step 325. If the operator elects to add the optional air or water, the process proceeds to step 320, wherein the operator may add the optional air or water in a variety of ways. For example, the operator may inject water into the mixture in an amount calculated to provide a desired degree of porosity. Alternatively, the operator may inject air into the mixture in an amount calculated to produce a desired degree of porosity. As still another example, the operator may permit air to become entrained into the mixture by pausing mixing for a desired period of time to permit entrainment of a desired amount of air, then resuming mixing after the desired degree of entrainment has occurred. Other means of enhancing the porosity of the mixture are possible, as may be recognized by one of ordinary skill in the art, with the benefit of this disclosure. Optionally, if an operator elects to reduce the porosity of the mixture, the operator may accomplish this, e.g., by vigorously stirring it in a downward motion in such fashion as to force air out of the mixture, thereby reducing the mixture's porosity. From step 320, the process proceeds to step 325, wherein the operator determines whether or not to add an optional catalyst to the mixture; such decision may be based on considerations including, inter alia, a desired reaction rate. If the operator elects not to add the optional catalyst, the process proceeds to step 335. If the operator elects to add the optional catalyst, the process proceeds to step 330 wherein the operator may mix in at least one catalyst (e.g., a tertiary amine). From step 330, the process proceeds to step 335, wherein the operator determines whether to mix in at least one filler material, surfactant, at least one radiotransparent substance, at least one radiopaque substance, and/or at least one protein. From step 335, the process proceeds to step 340, wherein isocyanate is mixed into the mixture, generally over a period of about 2 to about 3 minutes. In certain exemplary embodiments, the isocyanate and the first compound formed in step 310 both may be liquids at room temperature. From step 340, the process proceeds to step 345, wherein the operator determines whether the first compound and the isocyanate are reacting to form a polyester urethane at a desired rate. If the reaction is not proceeding at a desired rate, the process proceeds to step 350, wherein the operator enhances the reaction rate, e.g., by applying or removing heat. From step 350, the process returns to the determination in step 345, which previously has been described. If, however, the operator determines in step 345 that the reaction is proceeding at the desired rate, then the process proceeds to end.

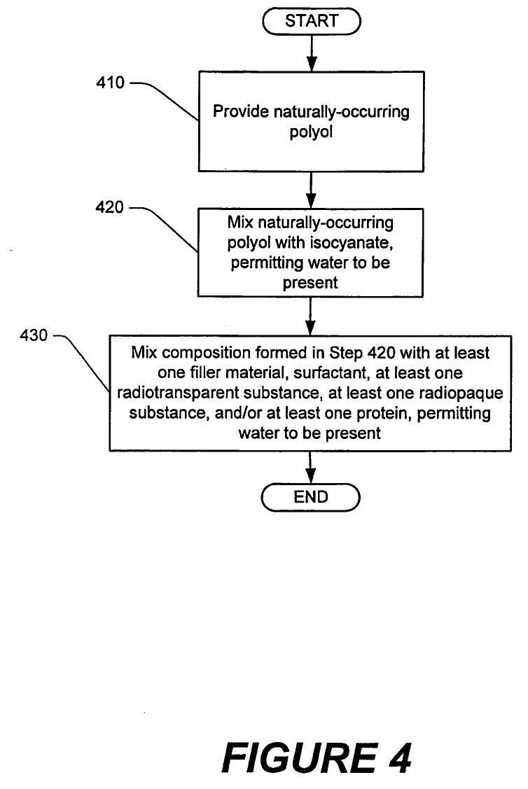

Referring now to FIG. 4, a naturally occurring polyol is provided in step 410. In step 420, the naturally occurring polyol may be mixed with an isocyanate, while permitting water to be present. In certain exemplary embodiments, the isocyanate and the naturally occurring polyol both may be liquids at room temperature. In an exemplary embodiment, in step 430, the mixture formed in step 420 further may be mixed with at least one filler material, a surfactant, at least one radio transparent substance, at least one radiopaque substance, and/or at least one protein. The process then proceeds to end. As an alternative, the additives added in step 430 may be added earlier, e.g., in a step (not shown) between step 410 and step 420, as opposed to after step 420; in such alternative embodiments, the process would proceed to end after step 420, the optional additives already having been added in a step (not shown) between step 410 and step 420.

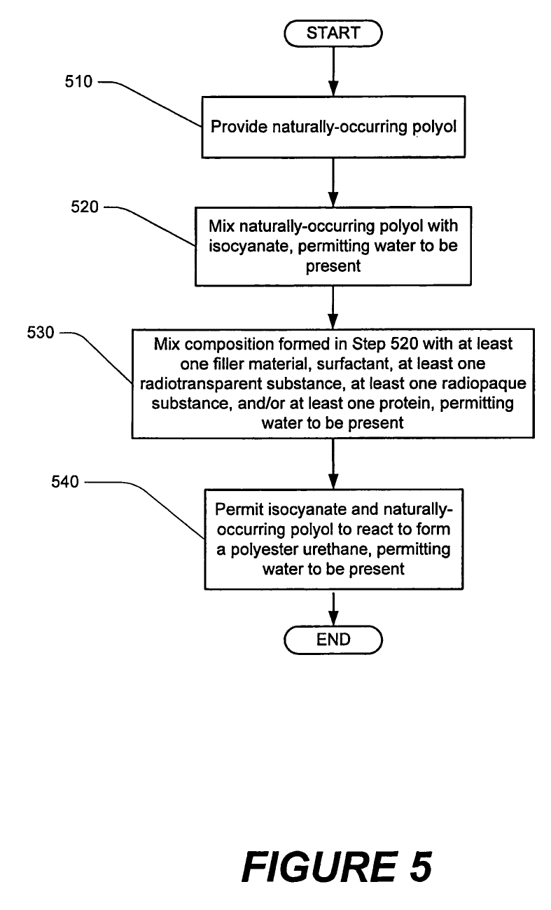

FIG. 5 illustrates an exemplary method for producing a composition of the present invention that comprises a polyester urethane. A naturally occurring polyol is provided in step 510. In step 520, the naturally occurring polyol may be mixed with an isocyanate, while permitting water to be present. In certain exemplary embodiments, the isocyanate and the naturally occurring polyol both may be liquids at room temperature. In an exemplary embodiment, in step 530, the mixture formed in step 520 further may be mixed with at least one filler material, a surfactant, at least one radio transparent substance, at least one radiopaque substance, and/or at least one protein. In step 540, the naturally occurring polyol and the isocyanate are permitted to react to form a polyester urethane, while permitting water to be present. The process then proceeds to end. As an alternative, the additives added in step 530 may be added earlier, e.g., in a step (not shown) between step 510 and step 520, as opposed to after step 520; in such alternative embodiments, the process would proceed to step 540 after step 520, the optional additives already having been added in a step (not shown) between step 510 and step 520.

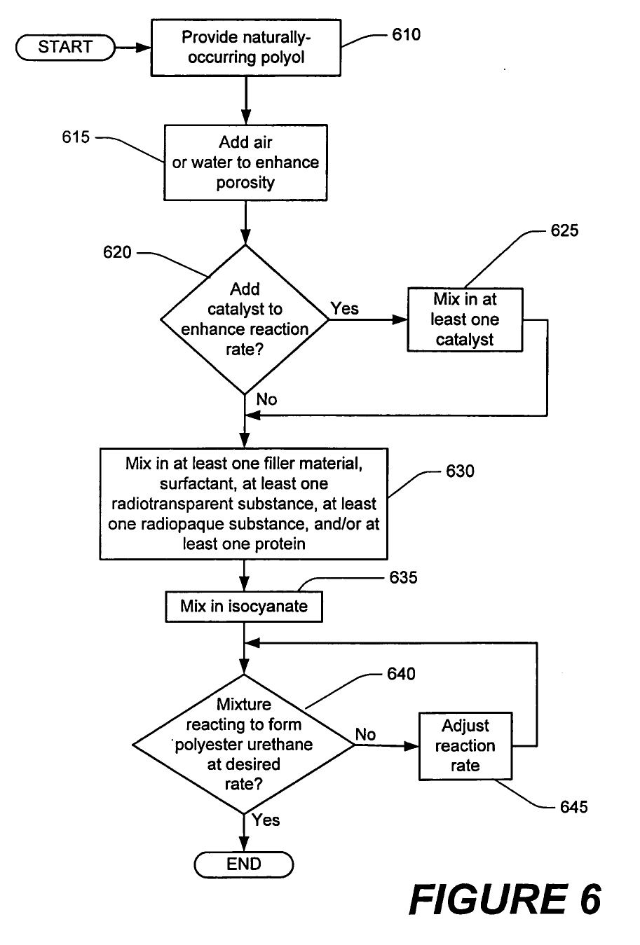

FIG. 6 illustrates an exemplary method for producing a composition of the present invention that comprises a polyester urethane. A naturally occurring polyol is provided in step 610. In step 615, the operator adds water or air to the mixture in an amount that may, inter alia, enhance the porosity of the final composition. The operator may add the air or water in a variety of ways. For example, the operator may inject water into the mixture in an amount calculated to provide a desired degree of porosity. Alternatively, the operator may inject air into the mixture in an amount calculated to produce a desired degree of porosity. As still another example, the operator may permit air to become entrained into the mixture by pausing mixing for a desired period of time to permit entrainment of a desired amount of air, then resuming mixing after the desired degree of entrainment has occurred. Other means of enhancing the porosity of the mixture are possible, as may be recognized by one of ordinary skill in the art, with the benefit of this disclosure. Optionally, if an operator elects to reduce the porosity of the mixture, the operator may accomplish this, e.g., by vigorously stirring it in a downward motion in such fashion as to force air out of the mixture, thereby reducing the mixture's porosity. From step 615, the process proceeds to step 620, wherein the operator determines whether or not to add an optional catalyst to the mixture; such decision may be based on considerations including, inter alia, a desired reaction rate. If the operator elects not to add the optional catalyst, the process proceeds to step 630. If the operator elects to add the optional catalyst, the process proceeds to step 625 wherein the operator may mix in at least one catalyst (e.g., a tertiary amine). From step 625, the process proceeds to step 630, wherein the operator determines whether to mix in at least one filler material, surfactant, at least one radiotransparent substance, at least one radiopaque substance, and/or at least one protein. From step 630, the process proceeds to step 635, wherein isocyanate is mixed into the mixture, generally over a period of about 2 to about 3 minutes. In certain exemplary embodiments, the isocyanate and the naturally occurring polyol both may be liquids at room temperature. From step 635, the process proceeds to step 640, wherein the operator determines whether the naturally occurring polyol and the isocyanate are reacting to form a polyester urethane at a desired rate. If the reaction is not proceeding at a desired rate, the process proceeds to step 645, wherein the operator enhances the reaction rate, e.g., by applying or removing heat. From step 645, the process returns to the determination in step 640, which previously has been described. If, however, the operator determines in step 640 that the reaction is proceeding at the desired rate, then the process proceeds to end.

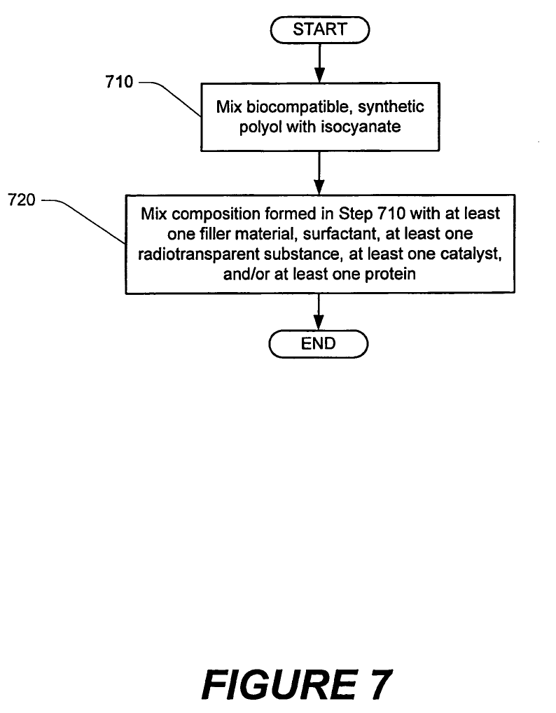

FIG. 7 illustrates another exemplary method of making a composition of the present invention. In step 710, a biocompatible, synthetic polyol may be mixed with an isocyanate. In certain exemplary embodiments of the present invention, the isocyanate and the biocompatible, synthetic polyol both may be liquids at room temperature. In an exemplary embodiment, in step 720, the composition formed in step 710 may be mixed with at least one filler material, a surfactant, at least one radio transparent substance, at least one radiopaque substance, and/or at least one protein. The process then proceeds to end. As an alternative, the additives added in step 720 may be added to the biocompatible, synthetic polyol earlier, e.g., in a step (not shown) before step 710, as opposed to after step 710; in such alternative embodiments, the process would proceed to end after step 710, the optional additives already having been added in a step (not shown) before step 710.

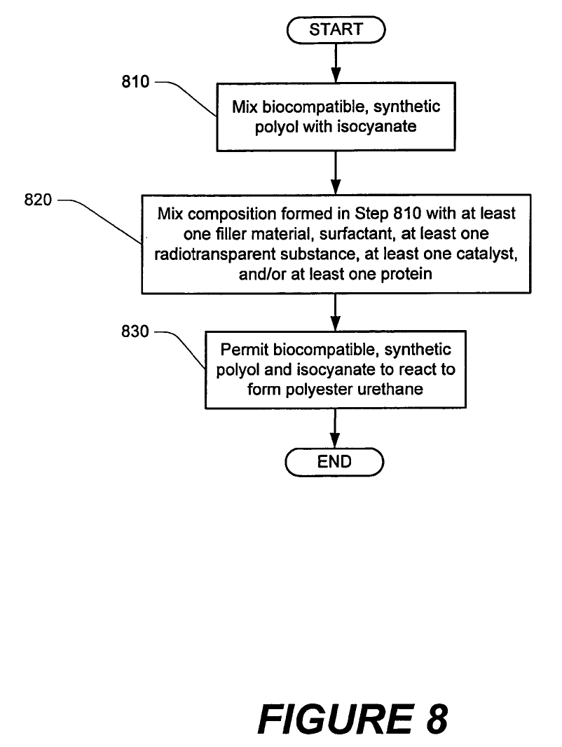

FIG. 8 illustrates an exemplary method of making a composition of the present invention that comprises a polyester urethane component. In step 810, a biocompatible, synthetic polyol may be mixed with an isocyanate. In certain exemplary embodiments, the isocyanate and the biocompatible, synthetic polyol both may be liquids at room temperature. In an exemplary embodiment, in step 820, the composition formed in step 810 may be mixed with at least one filler material, a surfactant, at least one radio transparent substance, at least one radiopaque substance, and/or at least one protein. In step 830, the biocompatible, synthetic polyol and the isocyanate are permitted to react to form a polyester urethane. The process then proceeds to end. As an alternative, the additives added in step 820 may be added to the biocompatible, synthetic polyol earlier, e.g., in a step (not shown) between before step 810, as opposed to after step 820; in such alternative embodiments, the process would proceed to step 830 after step 810, the optional additives already having been added in a step (not shown) before step 810.

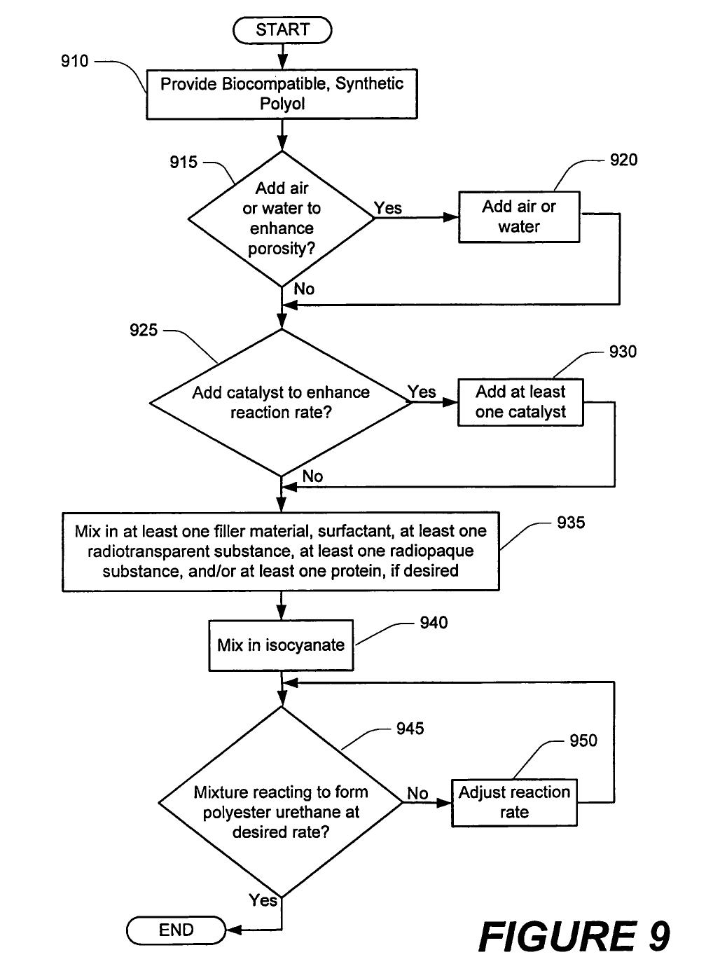

FIG. 9 illustrates another exemplary method of making a composition of the present invention that comprises a polyester urethane component. In step 910, a biocompatible, synthetic polyol is provided. In step 915, the operator determines whether or not to add air or water to the biocompatible, synthetic polyol in an amount that may, inter alia, enhance the porosity of the final composition. If the operator elects not to add the air or water, the process proceeds to step 925. If the operator elects to add the optional air or water, the process proceeds to step 920, wherein the operator may add the optional air or water in a variety of ways. For example, the operator may inject water into the mixture in an amount calculated to provide a desired degree of porosity. Alternatively, the operator may inject air into the mixture in an amount calculated to produce a desired degree of porosity. As still another example, the operator may permit air to become entrained into the mixture by pausing mixing for a desired period of time to permit entrainment of a desired amount of air, then resuming mixing after the desired degree of entrainment has occurred. Other means of enhancing the porosity of the mixture are possible, as may be recognized by one of ordinary skill in the art, with the benefit of this disclosure. Optionally, if an operator elects to reduce the porosity of the mixture, the operator may accomplish this, e.g., by vigorously stirring it in a downward motion in such fashion as to force air out of the mixture, thereby reducing the mixture's porosity. From step 920, the process proceeds to step 925, wherein the operator determines whether or not to add an optional catalyst to the mixture; such decision may be based on considerations including, inter alia, a desired reaction rate. If the operator elects not to add the optional catalyst, the process proceeds to step 935. If the operator elects to add the optional catalyst, the process proceeds to step 930 wherein the operator may mix in at least one catalyst (e.g., a tertiary amine). From step 930, the process proceeds to step 935, wherein the operator determines whether to mix in at least one filler material, surfactant, at least one radiotransparent substance, at least one radiopaque substance, and/or at least one protein. From step 935, the process proceeds to step 940, wherein isocyanate is mixed into the mixture, generally over a period of about 2 to about 3 minutes. In certain exemplary embodiments, the isocyanate and the biocompatible, synthetic polyol both may be liquids at room temperature. From step 940 the process proceeds to step 945, wherein the operator determines whether the isocyanate and the biocompatible, synthetic polyol are reacting to form a polyester urethane at a desired rate. If the reaction is not proceeding at the desired rate, the process proceeds to step 950, wherein the operator adjusts the reaction rate, e.g., by applying or removing heat. From step 950, the process returns to the determination in step 945, which previously has been described. If, however, the operator determines in step 945 that the reaction is proceeding at the desired rate, then the process proceeds to end.

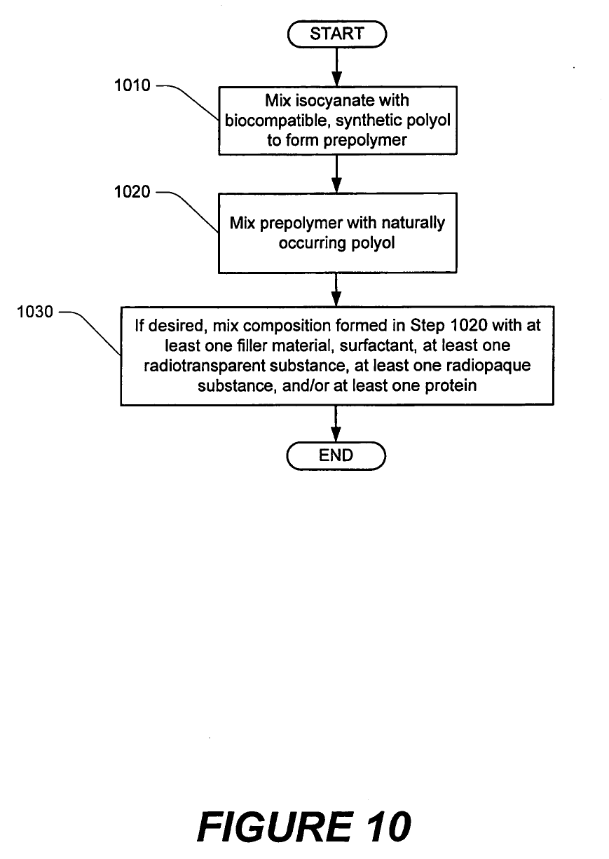

FIG. 10 illustrates still another exemplary method of making a composition of the present invention. In step 1010, a biocompatible, synthetic polyol may be mixed with an isocyanate to form an isocyanate prepolymer. In certain exemplary embodiments of the present invention, the isocyanate may become chemically bound within the isocyanate prepolymer to an extent sufficient to prevent the release of free isocyanate when the composition formed by this method is placed in the body of a mammal. In step 1020, the isocyanate prepolymer formed in step 1010 may be mixed with a naturally occurring polyol. In certain exemplary embodiments, the isocyanate prepolymer and the naturally occurring polyol both may be liquids at room temperature. In an exemplary embodiment, in step 1030, the composition formed in step 1020 may be mixed with at least one filler material, a surfactant, at least one radio transparent substance, at least one radiopaque substance, and/or at least one protein. The process then proceeds to end. As an alternative, the additives added in step 1030 may be added earlier, e.g., in a step (not shown) between step 1010 and step 1020, as opposed to after step 1020; in such alternative embodiments, the process would proceed to end after step 1020, the optional additives already having been added in a step (not shown) between step 1010 and step 1020.

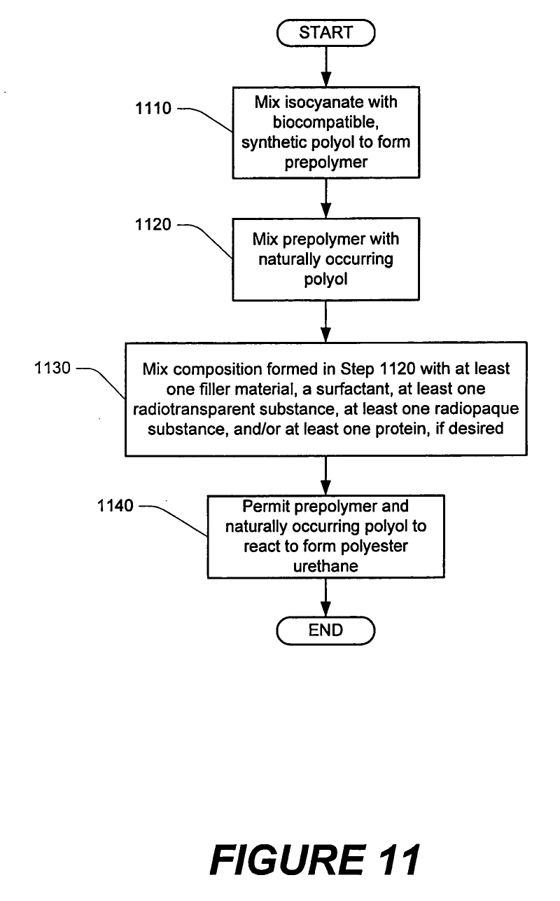

FIG. 11 illustrates still another exemplary method of making a composition of the present invention that comprises a polyester urethane. In step 1110, a biocompatible, synthetic polyol may be mixed with an isocyanate to form an isocyanate prepolymer. In certain exemplary embodiments of the present invention, the isocyanate may become chemically bound within the isocyanate prepolymer to an extent sufficient to prevent the release of free isocyanate when the composition formed by this method is placed in the body of a mammal. In step 1120, the isocyanate prepolymer formed in step 1110 may be mixed with a naturally occurring polyol. In certain exemplary embodiments, the isocyanate prepolymer and the naturally occurring polyol both may be liquids at room temperature. In an exemplary embodiment, in step 1130, the composition formed in step 1120 may be mixed with at least one filler material, a surfactant, at least one radio transparent substance, at least one radiopaque substance, and/or at least one protein. In step 1140, the isocyanate prepolymer and the naturally occurring polyol are permitted to react to form a polyester urethane. The process then proceeds to end. As an alternative, the additives added in step 1130 may be added earlier, e.g., in a step (not shown) between step 1110 and step 1120, as opposed to after step 1120; in such alternative embodiments, the process would proceed to step 1140 after step 1120, the optional additives already having been added in a step (not shown) between step 1110 and step 1120.

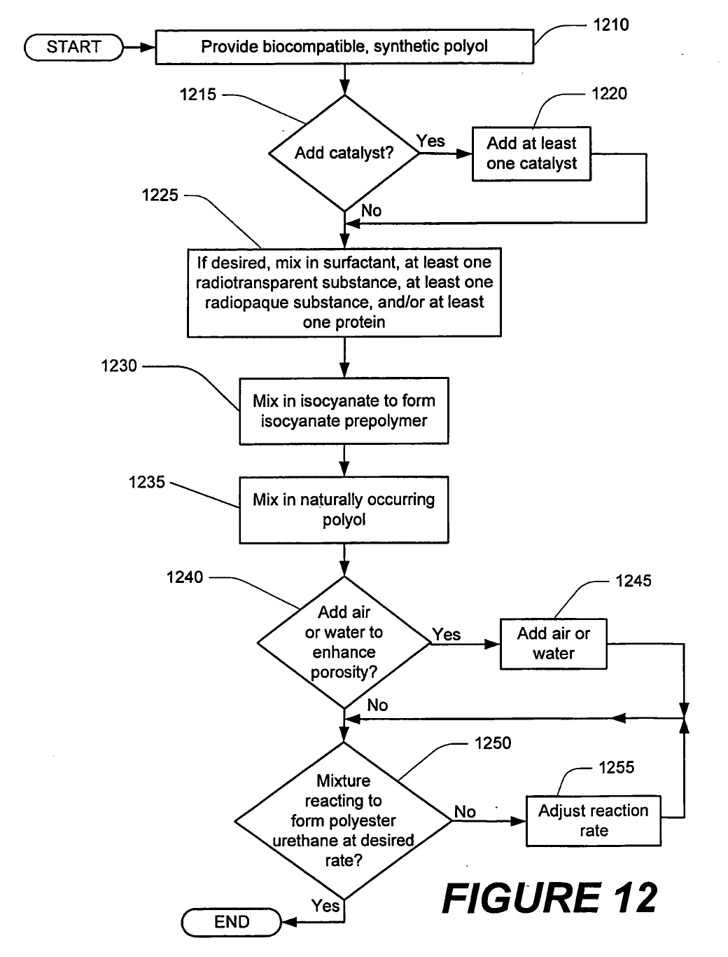

FIG. 12 illustrates still another exemplary method of making a composition of the present invention that comprises a polyester urethane. In step 1210, a biocompatible, synthetic polyol is provided. From step 1210, the process proceeds to step 1215, wherein the operator determines whether or not to add an optional catalyst to the mixture; such decision may be based on considerations including, inter alia, a desired reaction rate. If the operator elects not to add the optional catalyst, the process proceeds to step 1225. If the operator elects to add the optional catalyst, the process proceeds to step 1220 wherein the operator may mix in at least one catalyst (e.g., a tertiary amine). From step 1220, the process proceeds to step 1225, wherein the operator determines whether to mix in at least one filler material, surfactant, at least one radiotransparent substance, at least one radiopaque substance, and/or at least one protein. From step 1225, the process proceeds to step 1230, wherein isocyanate is mixed into the mixture, generally over a period of about 2 to about 3 minutes, so as to form an isocyanate prepolymer. In certain exemplary embodiments, the isocyanate and the biocompatible, synthetic polyol both may be liquids at room temperature. In certain exemplary embodiments of the present invention, the isocyanate may become chemically bound within the isocyanate prepolymer to an extent sufficient to prevent the release of free isocyanate when the composition formed by this method is placed in the body of a mammal. From step 1230, the process proceeds to step 1235, wherein a naturally occurring polyol and, optionally, at least one filler material are mixed into the mixture. In certain exemplary embodiments, the naturally occurring polyol and the isocyanate prepolymer both may be liquids at room temperature. In step 1240, the operator determines whether or not to add air or water to the biocompatible, synthetic polyol in an amount that may, inter alia, enhance the porosity of the final composition. If the operator elects not to add the air or water, the process proceeds to step 1250. If the operator elects to add the optional air or water, the process proceeds to step 1245, wherein the operator may add the optional air or water in a variety of ways. For example, the operator may inject water into the mixture in an amount calculated to provide a desired degree of porosity. Alternatively, the operator may inject air into the mixture in an amount calculated to produce a desired degree of porosity. As still another example, the operator may permit air to become entrained into the mixture by pausing mixing for a desired period of time to permit entrainment of a desired amount of air, then resuming mixing after the desired degree of entrainment has occurred. Other means of enhancing the porosity of the mixture are possible, as may be recognized by one of ordinary skill in the art, with the benefit of this disclosure. Optionally, if an operator elects to reduce the porosity of the mixture, the operator may accomplish this, e.g., by vigorously stirring it in a downward motion in such fashion as to force air out of the mixture, thereby reducing the mixture's porosity. From step 945, the process proceeds to step 1250, wherein the operator determines whether the isocyanate prepolymer and the naturally occurring polyol are reacting to form a polyester urethane at a desired rate. If the mixture is not reacting at a desired rate, the process proceeds to step 1255, wherein the operator enhances the reaction rate, e.g., by applying or removing heat. From step 1255, the process returns to the determination in step 1250, which previously has been previously described. If, however, the operator determines in step 1250 that the reaction is proceeding at the desired rate, then the process proceeds to end.

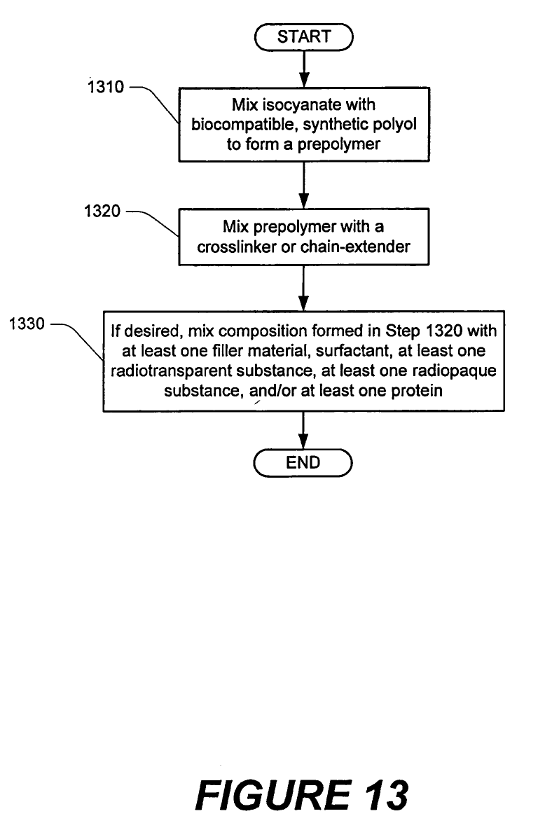

FIG. 13 illustrates another exemplary method of making a composition of the present invention. In step 1310, a biocompatible, synthetic polyol may be mixed with an isocyanate to form an isocyanate prepolymer. In certain exemplary embodiments of the present invention, the isocyanate may become chemically bound within the isocyanate prepolymer to an extent sufficient to prevent the release of free isocyanate when the composition formed by this method is placed in the body of a mammal. In step 1320, the isocyanate prepolymer formed in step 1310 may be mixed with a crosslinker or a chain extender; in certain exemplary embodiments, a crosslinker is used. In certain exemplary embodiments, the isocyanate prepolymer and the crosslinker or chain-extender both may be liquids at room temperature. In an exemplary embodiment, in step 1330, the composition formed in step 1320 may be mixed with at least one filler material, surfactant, at least one radio transparent substance, at least one radiopaque substance, and/or at least one protein. The process then proceeds to end. As an alternative, the additives added in step 1330 may be added earlier, e.g., in a step (not shown) between step 1310 and step 1320, as opposed to after step 1320; in such alternative embodiments, the process would proceed to end after step 1320, the optional additives already having been added in a step (not shown) between step 1310 and step 1320.

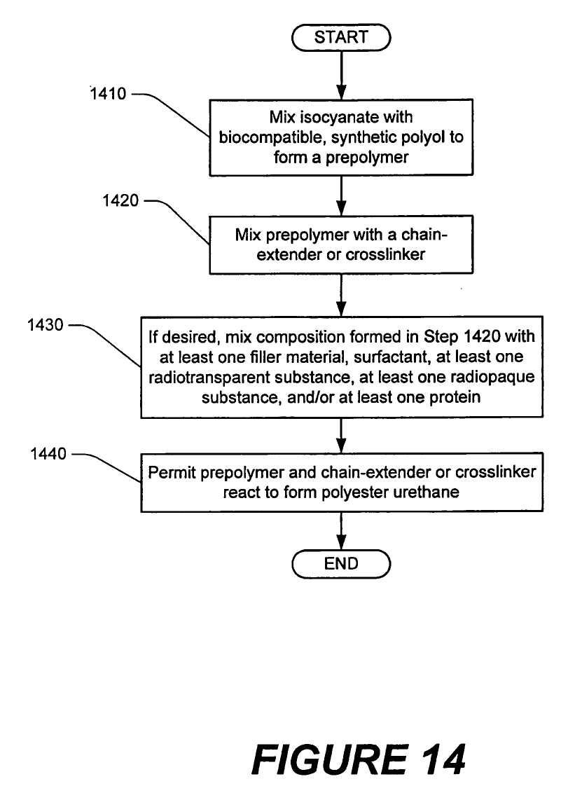

FIG. 14 illustrates another exemplary method of making a composition of the present invention comprising a polyester urethane. In step 1410, a biocompatible, synthetic polyol may be mixed with an isocyanate to form an isocyanate prepolymer. In certain exemplary embodiments of the present invention, the isocyanate may become chemically bound within the isocyanate prepolymer to an extent sufficient to prevent the release of free isocyanate when the composition formed by this method is placed in the body of a mammal. In step 1420, the isocyanate prepolymer formed in step 1410 may be mixed with a crosslinker or chain-extender; in certain exemplary embodiments, a crosslinker is used. In certain exemplary embodiments, the isocyanate prepolymer and the crosslinker both may be liquids at room temperature. In an exemplary embodiment, in step 1430, the composition formed in step 1420 may be mixed with at least one filler material, surfactant, at least one radio transparent substance, at least one radiopaque substance, and/or at least one protein. In step 1440, the isocyanate prepolymer and the vegetable oil are permitted to react to form a polyester urethane. The process then proceeds to end. As an alternative, the additives added in step 1430 may be added earlier, e.g., in a step (not shown) between step 1410 and step 1420, as opposed to after step 1420; in such alternative embodiments, the process would proceed to step 1440 after step 1420, the optional additives already having been added in a step (not shown) between step 1410 and step 1420.

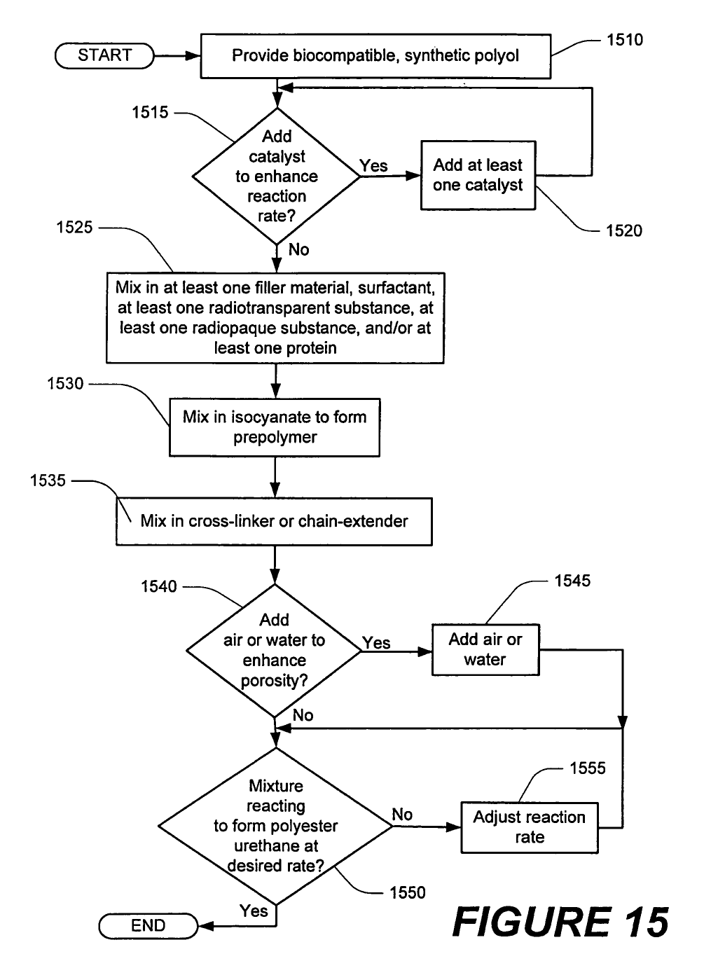

FIG. 15 illustrates still another exemplary method of making a composition of the present invention that comprises a polyester urethane. In step 1510, a biocompatible, synthetic polyol is provided. From step 1510, the process proceeds to step 1515, wherein the operator determines whether or not to add an optional catalyst to the mixture; such decision may be based on considerations including, inter alia, a desired reaction rate. If the operator elects not to add the optional catalyst, the process proceeds to step 1525. If the operator elects to add the optional catalyst, the process proceeds to step 1520 wherein the operator may mix in at least one catalyst (e.g., a tertiary amine). From step 1520, the process proceeds to step 1525, wherein the operator determines whether to mix in at least one filler material, surfactant, at least one radiotransparent substance, at least one radiopaque substance, and/or at least one protein. From step 1525, the process proceeds to step 1530, wherein isocyanate is mixed into the mixture, generally over a period of about 2 to about 3 minutes, so as to form an isocyanate prepolymer. In certain exemplary embodiments, the isocyanate and the biocompatible, synthetic polyol both may be liquids at room temperature. In certain exemplary embodiments of the present invention, the isocyanate may become chemically bound within the isocyanate prepolymer to an extent sufficient to prevent the release of free isocyanate when the composition formed by this method is placed in the body of a mammal. From step 1530, the process proceeds to step 1535, wherein a crosslinker or chain-extender and, optionally, at least one filler material are mixed into the mixture; in certain exemplary embodiments, a crosslinker is used. In certain exemplary embodiments, the crosslinker or chain-extender and the isocyanate prepolymer both may be liquids at room temperature. In step 1540, the operator determines whether or not to add air or water to the biocompatible, synthetic polyol in an amount that may, inter alia, enhance the porosity of the final composition. If the operator elects not to add the air or water, the process proceeds to step 1550. If the operator elects to add the optional air or water, the process proceeds to step 1545, wherein the operator may add the optional air or water in a variety of ways. For example, the operator may inject water into the mixture in an amount calculated to provide a desired degree of porosity. Alternatively, the operator may inject air into the mixture in an amount calculated to produce a desired degree of porosity. As still another example, the operator may permit air to become entrained into the mixture by pausing mixing for a desired period of time to permit entrainment of a desired amount of air, then resuming mixing after the desired degree of entrainment has occurred. Other means of enhancing the porosity of the mixture are possible, as may be recognized by one of ordinary skill in the art, with the benefit of this disclosure. Optionally, if an operator elects to reduce the porosity of the mixture, the operator may accomplish this, e.g., by vigorously stirring it in a downward motion in such fashion as to force air out of the mixture, thereby reducing the mixture's porosity. From step 1545, the process proceeds to step 1550, wherein the operator determines whether the isocyanate prepolymer and the crosslinker or chain-extender are reacting to form a polyester urethane at a desired rate. If the mixture is not reacting at a desired rate, the process proceeds to step 1555, wherein the operator enhances the reaction rate, e.g., by applying or removing heat. From step 1555, the process returns to the determination in step 1550, which previously has been previously described. If, however, the operator determines in step 1550 that the reaction is proceeding at the desired rate, then the process proceeds to end.

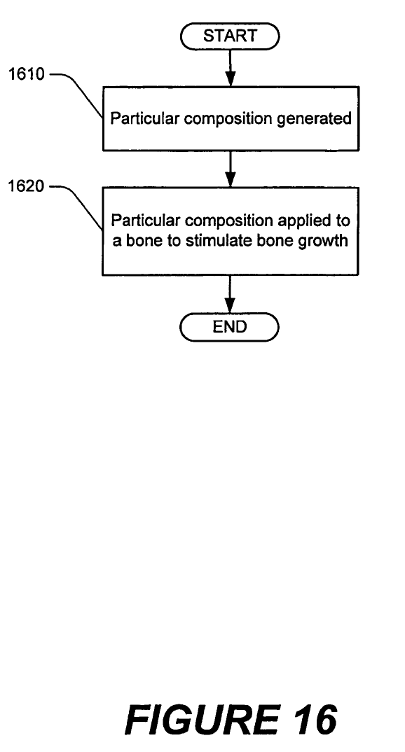

FIG. 16 depicts an exemplary embodiment of a method 1600 for performing a medical procedure, e.g., a non-invasive or an invasive medical procedure. In step 1610, a composition of the present invention may be generated, which composition may be the same or substantially the same as any of the compositions of the present invention. In certain exemplary embodiments, the composition may be a composition that comprises a polyester urethane. In step 1620, the composition of the present invention may be applied to a portion of a bone of a mammal. For example, a needle may be inserted through a skin of the mammal, and the composition may be dispensed onto a surface of the bone, e.g., at a location of a damaged portion of the bone, and the particular composition may stimulate bone growth.

While not willing to be bound by a theory, it is believed that cyclic adenosine monophosphate (cyclic AMP) regulated lipases within the body of a mammal may facilitate the metabolism of the compositions of the present invention after placement of these compositions in contact with, or in the vicinity of, a bone of the mammal. The compositions of the present invention that comprise a polyester urethane and that are placed in contact with, or in the vicinity of, a bone of a mammal generally comprise at least one ester group (e.g., a glyceride group) within their chemical structure. Water that naturally is present within the mammal then may react with the at least one ester group so as to be converted into glycerol, fatty acids, and the conversion of adenosine diphosphate to adenosine triphosphate. While not willing to be bound by theory, it is believed that adenosine triphosphate units within the mammal may support various anabolic activities that may result in the formation of bone.

When a composition of the present invention is placed in contact with, or in the vicinity of, a bone of a mammal, the composition may be a liquid, and may conform to a shape of the bone. The composition may transform into a solid after such placement within the mammal. In another embodiment of the present invention, method 1600 also may comprise the step of increasing or decreasing a temperature of the composition before or after the placement of the composition within the body of the mammal. Increasing the temperature of a composition of the present invention may decrease an amount of time which it takes for the particular composition to transform or cure from a liquid to a solid. Analogously, decreasing the temperature of a composition of the present invention may increase an amount of time which it takes for such transformation or curing of the composition to occur.

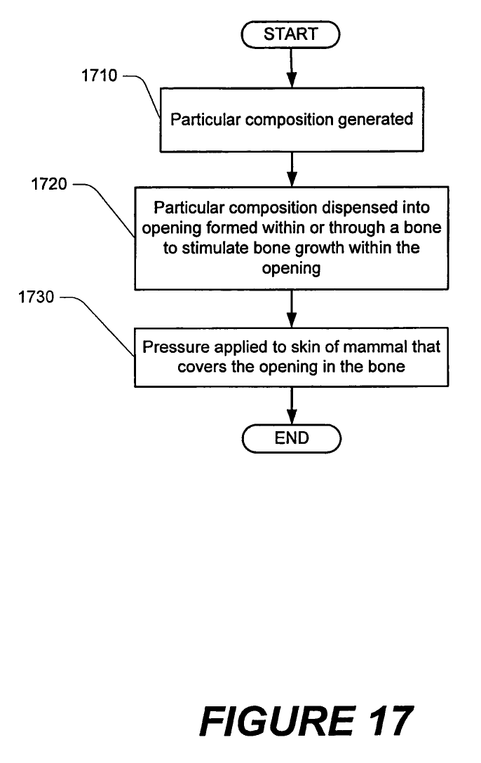

FIGS. 17 through 22 illustrate additional exemplary methods of the present invention for performing a medical procedure. Because certain features and advantages of these exemplary embodiments of the present invention are substantially similar to certain features and advantages of the above-described embodiments of the present invention, such similar features and advantages of the above-described embodiments of the present invention are not discussed further with respect to the exemplary embodiments of the present invention illustrated in FIGS. 17 through 22.

Referring now to FIG. 17, another exemplary embodiment of a method 1700 for performing a medical procedure, e.g., a non-invasive or an invasive medical procedure, is depicted therein. In step 1710, a composition of the present invention may be generated, which composition may be the same or substantially the same as any of the compositions of the present invention that have been set forth previously. In step 1720, the composition is dispensed into an opening formed within or through at least one portion of a bone of a mammal. In an exemplary embodiment, method 1700 also may comprise step 1730, wherein pressure is applied to a skin of the mammal that covers the opening in the bone, which may alter the shape of the composition of the present invention within the opening.

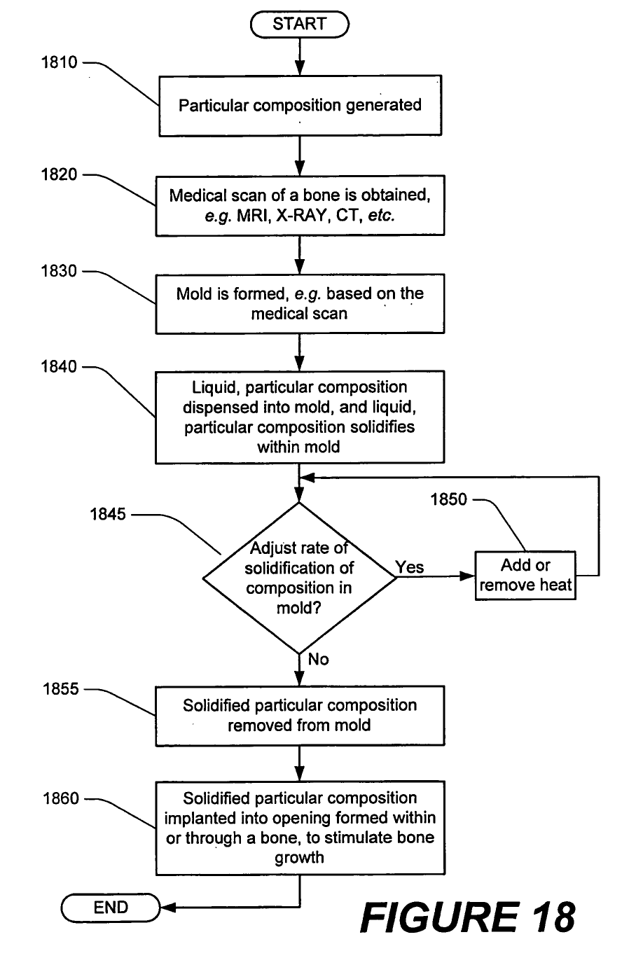

FIG. 18 illustrates still another exemplary embodiment of a method 1800 for performing a medical procedure, e.g., a non-invasive or an invasive medical procedure. In step 1810, a composition of the present invention may be generated, which may be the same or substantially the same as any of the compositions of the present invention set forth previously. In step 1820, a medical scan of a bone of a mammal may be obtained, e.g., a CT scan, an MRI scan, an X-ray scan, or the like. In step 1830, a mold may be formed, e.g., based on the medical scan or based on a generic size for the mold, and in step 1840, the liquid composition of the present invention may be dispensed into the mold, and permitted to solidify therein. In step 1845, the operator determines whether or not to adjust the rate at which the composition solidifies within the mold. If the operator elects to adjust the rate of solidification, the process proceeds to step 1850, wherein the operator adjusts the rate of solidification, e.g., by adding or removing heat. Generally, the addition of heat will increase the rate of solidification, whereas the removal of heat will decrease the rate of solidification. From step 1850, the process returns to the determination in step 1845. If, in step 1845, the operator elects not to adjust the rate of solidification, the process proceeds to step 1855. In step 1855, the solidified composition may be removed from the mold, and in step 1860, the solidified composition may be implanted into an opening formed within or through at least one portion of the bone.

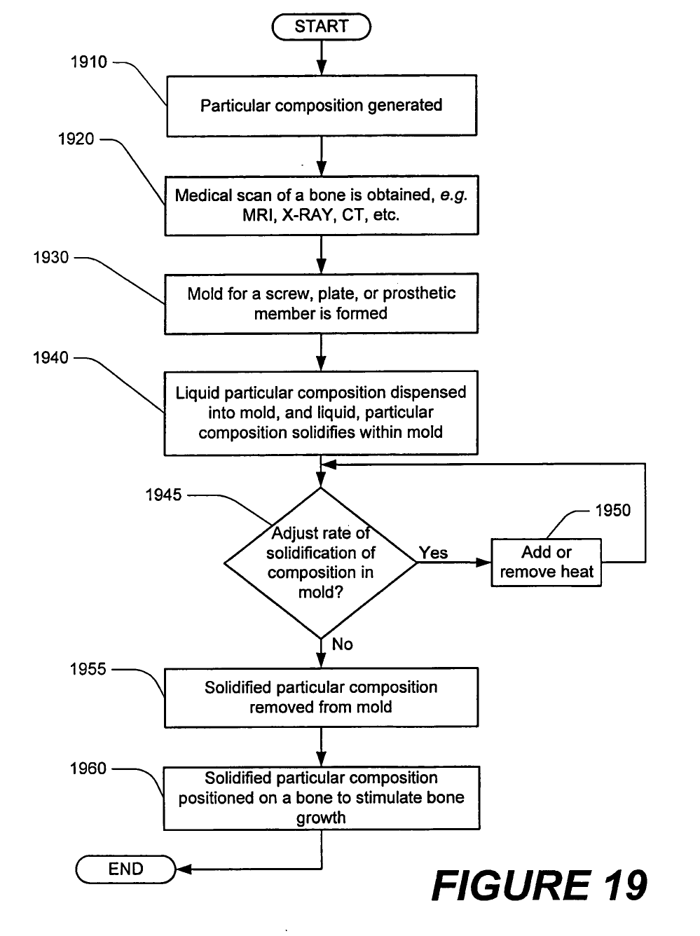

FIG. 19 illustrates another exemplary embodiment of a method 1900 for performing a medical procedure, e.g., a non-invasive or an invasive medical procedure. In step 1910, a composition of the present invention may be generated, which may be the same or substantially the same as any of the compositions of the present invention set forth previously. In step 1920, a medical scan of a bone of a mammal may be obtained, e.g., a CT scan, an MRI scan, an X-ray scan, or the like. In step 1930, a mold may be formed, e.g., based on the medical scan or based on a generic size for the mold, and in step 1940, the liquid composition may be dispensed into the mold, and permitted to solidify therein. In step 1945, the operator determines whether or not to adjust the rate at which the composition solidifies within the mold. If the operator elects to adjust the rate of solidification, the process proceeds to step 1950, wherein the operator adjusts the rate of solidification, e.g., by adding or removing heat. Generally, the addition of heat will increase the rate of solidification, whereas the removal of heat will decrease the rate of solidification. From step 1950, the process returns to the determination in step 1945. If, in step 1945, the operator elects not to adjust the rate of solidification, the process proceeds to step 1955. In step 1955, the solidified composition of the present invention may be removed from the mold, and in step 1960, the solidified composition of the present invention may be positioned on at least one portion of the bone.

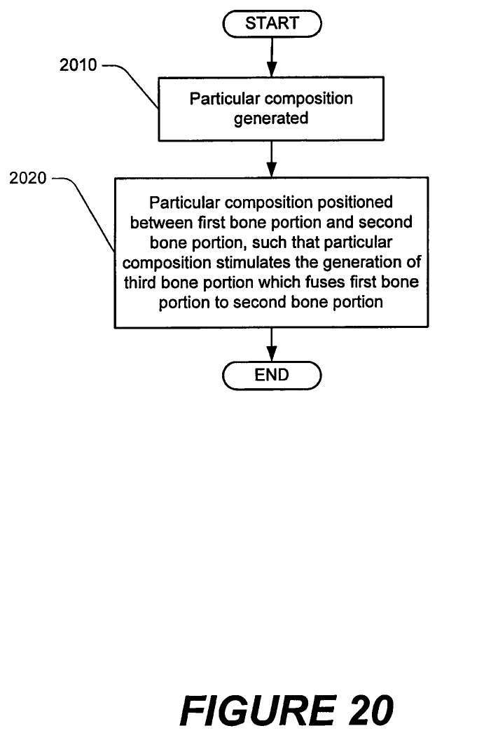

FIG. 20 illustrates still another exemplary embodiment of a method 2000 for performing a medical procedure, e.g., a non-invasive or an invasive medical procedure. In step 2010, a particular composition may be generated, which may be the same or substantially the same as any of the compositions of the present invention that have been set forth previously. In step 2020, the composition may be positioned between a first bone portion of a mammal and a second bone portion of the mammal for fusing the first bone portion to the second bone portion, such that the composition stimulates the growth of a third bone portion that fuses the first bone portion to the second bone portion. For example, the composition may be injected into a balloon, and the balloon may be positioned between the first bone portion and the second bone portion. In an exemplary embodiment, the balloon may rest on tissue of the mammal, and the tissue may degrade the balloon before the composition solidifies. Moreover, the same bone within the mammal may comprise each of the first bone portion and the second bone portion, or a first bone may comprise the first bone portion and a second bone may comprise the second bone portion. For example, the first bone may be a first vertebra of a spine of the mammal and the second bone may be a second vertebra of the spine.

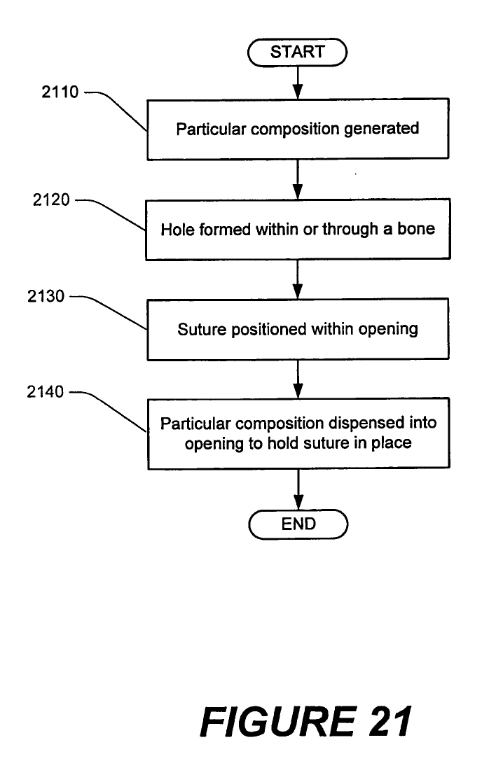

FIG. 21 depicts another exemplary embodiment of a method 2100 for performing a medical procedure, e.g., a non-invasive or an invasive medical procedure. In step 2110, a composition may be generated, which may be the same or substantially the same as any of the compositions of the present invention that have been set forth previously. In step 2120, a hole in a bone of a mammal may be formed, e.g., drilled, within or through the bone. In step 2130, at least one suture may be positioned within the opening formed within or through the bone. For example, a fluid or a powder which prevents the suture from adhering to the composition of the present invention may be applied to the suture, and then the suture may be dispensed in the opening. In step 2140, the composition of the present invention may be dispensed into the opening to prevent the suture from falling out of the opening.

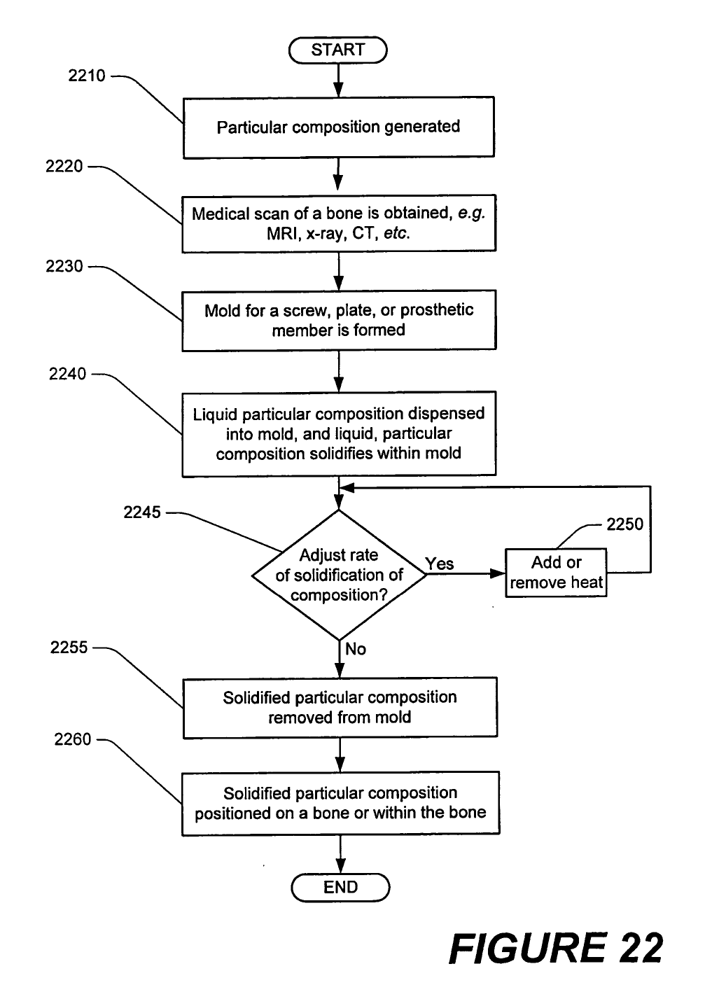

FIG. 22 depicts another exemplary embodiment of a method 2200 for performing a medical procedure, e.g., a non-invasive or an invasive medical procedure. In step 2210, a composition may be generated, which may be the same or substantially the same as any of the compositions of the present invention set forth previously. In step 2220, a medical scan of a bone of a mammal may be obtained, e.g., a CT scan, an MRI scan, an X-ray scan, or the like. In step 2230, a mold may be formed, which mold may comprise a mold for a screw, a mold for a plate, a mold for a prosthetic member, or the like. In step 2240, the liquid composition may be dispensed into the mold, and permitted to solidify therein. In step 2245, the operator determines whether or not to adjust the rate at which the composition solidifies within the mold. If the operator elects to adjust the rate of solidification, the process proceeds to step 2250, wherein the operator adjusts the rate of solidification, e.g., by adding or removing heat. Generally, the addition of heat will increase the rate of solidification, whereas the removal of heat will decrease the rate of solidification. From step 2250, the process returns to the determination in step 2245. If, in step 2245, the operator elects not to adjust the rate of solidification, the process proceeds to step 2255. In step 2255, the solidified composition of the present invention may be removed from the mold, and in step 2260, the solidified composition of the present invention may be positioned on a bone of a mammal or within an opening formed within the bone.

According to an exemplary embodiment of the present invention, a kit that is useful in promoting bone growth comprises a first container that further comprises a dispensing means and a first compound, and a second container that further comprises a dispensing means and a second compound. In certain exemplary embodiments, the first container and the second container each may be a syringe. In certain exemplary embodiments, the first container and the second container may be packaged together in a moisture resistant package, such that the first compound and the second compound may be maintained in the same package, but separated from each other. The first compound and the second compound may be readily combined to form a composition of the present invention at a desired time before the performance of a medical procedure by dispensing the first compound and the second compound from their respective containers. In certain exemplary embodiments, the first compound may comprise a naturally occurring polyol. In certain of such exemplary embodiments, the second compound may comprise an isocyanate prepolymer comprising isocyanate. In certain exemplary embodiments, the first compound may comprise a biocompatible, synthetic polyol. In certain of such exemplary embodiments, the second compound may comprise isocyanate. In certain exemplary embodiments, the first compound may comprise a polyol comprising a naturally occurring polyol and a biocompatible synthetic polyol. In certain of such exemplary embodiments, the second compound may comprise isocyanate.

To facilitate a better understanding of the present invention, the following examples of some exemplary embodiments are given. In no way should such examples be read to limit, or to define, the scope of the invention.

### EXAMPLE 1

A sample composition of the present invention was prepared by mixing RUBINATE 9433 with CASPOL® 5001 in a 4:1 equivalent ratio to form an isocyanate prepolymer. Calcium carbonate then was added to the isocyanate prepolymer in an amount of 72.4% by weight of the isocyanate prepolymer, and mixed for one minute to form a paste. CASPO® 1962 (containing DABCO 33LV in the amount of 0.20% by weight of the CASPOL® 1962) then was added to the isocyanate prepolymer and calcium carbonate mixture in the amount of 68.65% by weight of the isocyanate prepolymer, and mixed for 2 minutes in a plastic beaker with a spatula. To prepare test samples for hardness testing, a portion of the above mixture was poured into a cylindrical cavity (dimensions 1 in² by ½ inch) in a Teflon-coated mold, and visually observed for evidence of gelation. Once gelation was detected, the mold was covered with a Teflon-coated plate and placed into a Carver press under slight pressure. Hardness testing was performed according to ASTM 2240.

To prepare test samples for flexural property testing, a portion of the above mixture was poured at its gel time into a cavity of a rectangular aluminum mold (1.5 mm thick) that was coated with a Teflon sheet. Once gelation was detected, the mold was covered with a Teflon-coated plate, placed into a Carver press, and compression molded at about 20,000 psi. The test samples were cut by a saw. Flexural properties were tested according to ASTM D 790 by using an Instron Tester Model 1122 and Merlin software.

The results of the testing are set forth in the table below.

TABLE 1

| Properties | Sample Composition No. 1 |
|---|---|
| Shore D Hardness | 72 |
| Flexural Strength | 57.4 MPa |
| Strain at Yield | 5.8 |
| Modulus | 2031 MPa |

The above example demonstrates, inter alia, that the compositions of the present invention have mechanical properties that may be suitable for bone cements.

### EXAMPLE 2

Sample Composition No. 2, a sample composition of the present invention, was prepared by mixing ISONATE 50 OP with CASPOL® 5001 in a 4:1 equivalent ratio to form an isocyanate prepolymer. Calcium carbonate then was added to the isocyanate prepolymer in an amount of 72.4% by weight of the isocyanate prepolymer, and mixed for one minute to form a paste. CASPOL® 1962 (containing DABCO 33LV in the amount of 0.35% by weight of the CASPOL® 1962) then was added to the isocyanate prepolymer in the amount of 68.5% by weight of the isocyanate prepolymer, and mixed for 2 minutes in a plastic beaker with a spatula.

To prepare test samples for hardness testing, a portion of the above mixture was poured into a cylindrical cavity (dimensions 1 in² by 1/2 inch) in a Teflon-coated mold, and visually observed for evidence of gelation. Once gelation was detected, the mold was covered with a Teflon-coated plate and placed into a Carver press under slight pressure. Hardness testing was performed according to ASTM 2240.

To prepare test samples for flexural property testing, a portion of the above mixture was poured at its gel time into a cavity of a rectangular aluminum mold (1.5 mm thick) that was coated with a Teflon sheet. Once gelation was detected, the mold was covered with a Teflon-coated plate, placed into a Carver press, and compression molded at about 20,000 psi. The test samples were cut by a saw. Flexural properties were tested according to ASTM D 790 by using an Instron Tester Model 1122 and Merlin software.

The results of the testing are set forth in the table below.

TABLE 2

| Properties | Sample Composition No. 2 |
|---|---|
| Shore D Hardness | 82 |
| Flexural Strength | 84.9 MPa |
| Strain at Yield | 5.9 |
| Modulus | 3193 MPa |

The above example demonstrates, inter alia, that the compositions of the present invention have mechanical properties that may be suitable for bone cements.

While the invention has been described in connection with preferred embodiments, it will be understood by those of ordinary skill in the art that other variations and modifications of the preferred embodiments described above may be made without departing from the scope of the invention. Other embodiments will be apparent to those of ordinary skill in the art from a consideration of the specification or practice of the invention disclosed herein. It is intended that the specification and the described examples are considered as exemplary only, with the true scope and spirit of the invention indicated by the following claims.

1. A composition, comprising:
a polyol comprising:
a naturally occurring polyol; and
a biocompatible, synthetic polyol; and
isocyanate.

2. The composition of claim 1, further comprising at least one filler material selected from the group consisting of calcium carbonate, bone, calcium phosphate, calcium pyrophosphate, hydroxyapatite, poly methyl methacrylate, glass-ionomer, poly ether ether ketone, calcium sulfate, and tricalcium phosphate.

3. The composition of claim 2, wherein the at least one filler material is present in an amount in the range of from about 0.01% to about 30% by weight.

4. The composition of claim 2, wherein the at least one filler material is bone that is selected from the group consisting of demineralized bone, allograft bone, and autogenous bone.

5. The composition of claim 2, wherein the at least one filler material is beta tricalcium phosphate.

6. The composition of claim 1 wherein the isocyanate is selected from the group consisting of: an aromatic isocyanate, an aliphatic isocyanate, and a cycloaliphatic isocyanate.

7. The composition of claim 6 wherein the aromatic isocyanate is diphenylmethane diisocyanate.

8. The composition of claim 1 wherein the isocyanate is a cycloaliphatic isocyanate.

9. The composition of claim 1 wherein the naturally occurring polyol has at least one hydroxyl group.

10. The composition of claim 9 wherein the naturally occurring polyol is selected from the group consisting of castor oil, safflower oil, lesquerella oil, a polyol obtained by chemical modification of a naturally occurring vegetable oil, a naturally occurring oil that has been trans-esterified and a naturally occurring oil that has been hydrogenated.

11. The composition of claim 1 wherein the biocompatible, synthetic polyol is selected from the group consisting of: a polycaprolactone polyol, a polyester polyol, a polyadipate polyol, and a polyol derived from a synthetic acid.

12. The composition of claim 1 further comprising water.

13. The composition of claim 12 wherein the water is present in an amount sufficient to impart a desired degree of porosity to the composition.

14. The composition of claim 12 wherein the water is present in an amount in the range of from about 0.1% to about 1% by weight.

15. The composition of claim 13 having an average porosity in the range of from about 5 microns to about 500 microns.

16. The composition of claim 1, further comprising at least one substance selected from the group consisting of a radiotransparent substance and a radiopaque substance.

17. The composition of claim 16, wherein the at least one substance is at least one radiotransparent substance selected from the group consisting of gas, air, nitrogen gas, carbon dioxide, and oxygen gas.

18. The composition of claim 16, wherein the at least one substance is at least one radiopaque substance selected from the group consisting of insoluble zirconium oxide, a radioactive tracer, a Barium Sulfate contrast media, a gadolinium contrast media, a water-soluble Iodinated contrast media, an oily Iodinated contrast media, and an implantable metal.

19. The composition of claim 1, further comprising at least one protein.

20. The composition of claim 1, wherein the composition further comprises at least one catalyst.

21. The composition of claim 20, wherein the at least one catalyst comprises tertiary amine, or an organometallic compound.

22. The composition of claim 1, further comprising surfactant.

23. The composition of claim 1, wherein the composition is adapted to stimulate bone growth when the composition contacts or is positioned in the vicinity of a bone of a mammal.

24. A composition, comprising:
a naturally occurring polyol;
isocyanate; and
water.

25. The composition of claim 24, further comprising at least one filler material selected from the group consisting of calcium carbonate, bone, calcium phosphate, calcium pyrophosphate, hydroxyapatite, poly methyl methacrylate, glass-ionomer, poly ether ether ketone, calcium sulfate, and tricalcium phosphate.

26. The composition of claim 5, wherein the at least one filler material is present in an amount in the range of from about 0.01% to about 30% by weight.

27. The composition of claim 25, wherein the at least one filler material is bone that is selected from the group consisting of demineralized bone, allograft bone, and autogenous bone.

28. The composition of claim 25, wherein the at least one filler material is beta tricalcium phosphate.

29. The composition of claim 24 wherein the isocyanate is selected from the group consisting of: an aromatic isocyanate, an aliphatic isocyanate, and a cycloaliphatic isocyanate.

30. The composition of claim 29 wherein the aromatic isocyanate is diphenylmethane diisocyanate.

31. The composition of claim 24 wherein the isocyanate is a cycloaliphatic isocyanate.

32. The composition of claim 24 wherein the naturally occurring polyol has at least one hydroxyl group.

33. The composition of claim 24 wherein the naturally occurring polyol is selected from the group consisting of castor oil, safflower oil, lesquerella oil, a polyol obtained by chemical modification of a naturally occurring vegetable oil, a naturally occurring oil that has been trans-esterified and a naturally occurring oil that has been hydrogenated.

34. The composition of claim 24 wherein the biocompatible, synthetic polyol is selected from the group consisting of: a polycaprolactone polyol, a polyester polyol, a polyadipate polyol, and a polyol derived from a synthetic acid.

35. The composition of claim 24 wherein the water is present in an amount sufficient to impart a desired degree of porosity to the composition.

36. The composition of claim 24 wherein the water is present in an amount in the range of from about 0.1% to about 1% by weight.

37. The composition of claim 35 having an average porosity in the range of from about 5 microns to about 500 microns.

38. The composition of claim 24, further comprising at least one substance selected from the group consisting of a radiotransparent substance and a radiopaque substance.

39. The composition of claim 38, wherein the at least one substance is at least one radiotransparent substance selected from the group consisting of gas, air, nitrogen gas, carbon dioxide, and oxygen gas.

40. The composition of claim 38, wherein the at least one substance is at least one radiopaque substance selected from the group consisting of insoluble zirconium oxide, a radioactive tracer, a Barium Sulfate contrast media, a gadolinium contrast media, a water-soluble Iodinated contrast media, an oily Iodinated contrast media, and an implantable metal.

41. The composition of claim 24, further comprising at least one protein.

42. The composition of claim 24, wherein the composition further comprises at least one catalyst.

43. The composition of claim 42, wherein the at least one catalyst comprises tertiary amine, or an organometallic compound.

44. The composition of claim 24, further comprising surfactant.

45. The composition of claim 24, wherein the composition is adapted to stimulate bone growth when the composition contacts or is positioned in the vicinity of a bone of a mammal.

46. A composition, comprising:
a biocompatible, synthetic polyol; and
isocyanate.

47. The composition of claim 46, further comprising at least one filler material selected from the group consisting of calcium carbonate, bone, calcium phosphate, calcium pyrophosphate, hydroxyapatite, poly methyl methacrylate, glass-ionomer, poly ether ether ketone, calcium sulfate, and tricalcium phosphate.

48. The composition of claim 47, wherein the at least one filler material is present in an amount in the range of from about 0.01% to about 30% by weight.

49. The composition of claim 47, wherein the at least one filler material is bone that is selected from the group consisting of demineralized bone, allograft bone, and autogenous bone.

50. The composition of claim 47, wherein the at least one filler material is beta tricalcium phosphate.

51. The composition of claim 46 wherein the isocyanate is selected from the group consisting of: an aromatic isocyanate, an aliphatic isocyanate, and a cycloaliphatic isocyanate.

52. The composition of claim 51 wherein the aromatic isocyanate is diphenylmethane diisocyanate.

53. The composition of claim 46 wherein the isocyanate is a cycloaliphatic isocyanate.

54. The composition of claim 46 further comprising water.

55. The composition of claim 54 wherein the water is present in an amount sufficient to impart a desired degree of porosity to the composition.

56. The composition of claim 54 wherein the water is present in an amount in the range of from about 0.1% to about 1% by weight.

57. The composition of claim 55 having an average porosity in the range of from about 5 microns to about 500 microns.

58. The composition of claim 46, further comprising at least one substance selected from the group consisting of a radiotransparent substance and a radiopaque substance.

59. The composition of claim 58, wherein the at least one substance is at least one radiotransparent substance selected from the group consisting of gas, air, nitrogen gas, carbon dioxide, and oxygen gas.

60. The composition of claim 58, wherein the at least one substance is at least one radiopaque substance selected from the group consisting of insoluble zirconium oxide, a radioactive tracer, a Barium Sulfate contrast media, a gadolinium contrast media, a water-soluble Iodinated contrast media, an oily Iodinated contrast media, and an implantable metal.

61. The composition of claim 46, further comprising at least one protein.

62. The composition of claim 46, further comprising surfactant.

63. The composition of claim 46, wherein the composition is adapted to stimulate bone growth when the composition contacts or is positioned in the vicinity of a bone of a mammal.

64. A composition, comprising:
a naturally occurring polyol;
an isocyanate prepolymer comprising:
a biocompatible, synthetic polyol; and
isocyanate.

65. The composition of claim 64, further comprising at least one filler material selected from the group consisting of calcium carbonate, bone, calcium phosphate, calcium pyrophosphate, hydroxyapatite, poly methyl methacrylate, glass-ionomer, poly ether ether ketone, calcium sulfate, and tricalcium phosphate.

66. The composition of claim 65, wherein the at least one filler material is present in an amount in the range of from about 0.01% to about 30% by weight.

67. The composition of claim 65, wherein the at least one filler material is bone that is selected from the group consisting of demineralized bone, allograft bone, and autogenous bone.

68. The composition of claim 65, wherein the at least one filler material is beta tricalcium phosphate.

69. The composition of claim 64 wherein the isocyanate is selected from the group consisting of: an aromatic isocyanate, an aliphatic isocyanate, and a cycloaliphatic isocyanate.

70. The composition of claim 69 wherein the aromatic isocyanate is diphenylmethane diisocyanate.

71. The composition of claim 64 wherein the isocyanate is a cycloaliphatic isocyanate.

72. The composition of claim 64 wherein the biocompatible, synthetic polyol is selected from the group consisting of: a polycapralactone polyol, a polyester polyol, a polyadipate polyol, and a polyol derived from a synthetic acid.

73. The composition of claim 64 further comprising water.

74. The composition of claim 73 wherein the water is present in an amount sufficient to impart a desired degree of porosity to the composition.

75. The composition of claim 73 wherein the water is present in an amount in the range of from about 0.1% to about 1% by weight.

76. The composition of claim 74 having an average porosity in the range of from about 5 microns to about 500 microns.

77. The composition of claim 64, further comprising at least one substance selected from the group consisting of a radiotransparent substance and a radiopaque substance.

78. The composition of claim 77, wherein the at least one substance is at least one radiotransparent substance selected from the group consisting of gas, air, nitrogen gas, carbon dioxide, and oxygen gas.

79. The composition of claim 77, wherein the at least one substance is at least one radiopaque substance selected from the group consisting of insoluble zirconium oxide, a radioactive tracer, a Barium Sulfate contrast media, a gadolinium contrast media, a water-soluble Iodinated contrast media, an oily Iodinated contrast media, and an implantable metal.

80. The composition of claim 64, further comprising at least one protein.

81. The composition of claim 64, wherein the composition further comprises at least one catalyst.

82. The composition of claim 81, wherein the at least one catalyst comprises tertiary amine, or an organometallic compound.

83. The composition of claim 64, further comprising surfactant.

84. The composition of claim 64, wherein the composition is adapted to stimulate bone growth when the composition contacts or is positioned in the vicinity of a bone of a mammal.

85. A composition, comprising:
a crosslinker or chain-extender; and
an isocyanate prepolymer comprising:
a biocompatible, synthetic polyol; and
isocyanate.

86. The composition of claim 85, further comprising at least one filler material selected from the group consisting of calcium carbonate, bone, calcium phosphate, calcium pyrophosphate, hydroxyapatite, poly methyl methacrylate, glass-ionomer, poly ether ether ketone, calcium sulfate, and tricalcium phosphate.

87. The composition of claim 86, wherein the at least one filler material is present in an amount in the range of from about 0.01% to about 30% by weight.

88. The composition of claim 86, wherein the at least one filler material is bone that is selected from the group consisting of demineralized bone, allograft bone, and autogenous bone.

89. The composition of claim 86, wherein the at least one filler material is beta tricalcium phosphate.

90. The composition of claim 85 wherein the isocyanate is selected from the group consisting of: an aromatic isocyanate, an aliphatic isocyanate, and a cycloaliphatic isocyanate.

91. The composition of claim 90 wherein the aromatic isocyanate is diphenylmethane diisocyanate.

92. The composition of claim 85 wherein the isocyanate is a cycloaliphatic isocyanate.

93. The composition of claim 85 wherein the biocompatible, synthetic polyol is selected from the group consisting of: a polycapralactone polyol, a polyester polyol, a polyadipate polyol, and a polyol derived from a synthetic acid.

94. The composition of claim 85 further comprising water.

95. The composition of claim 94 wherein the water is present in an amount sufficient to impart a desired degree of porosity to the composition.

96. The composition of claim 94 wherein the water is present in an amount in the range of from about 0.1% to about 1% by weight.

97. The composition of claim 94 having an average porosity in the range of from about 5 microns to about 500 microns.

98. The composition of claim 85, further comprising at least one substance selected from the group consisting of a radiotransparent substance and a radiopaque substance.

99. The composition of claim 98, wherein the at least one substance is at least one radiotransparent substance selected from the group consisting of gas, air, nitrogen gas, carbon dioxide, and oxygen gas.

100. The composition of claim 98, wherein the at least one substance is at least one radiopaque substance selected from the group consisting of insoluble zirconium oxide, a radioactive tracer, a Barium Sulfate contrast media, a gadolinium contrast media, a water-soluble Iodinated contrast media, an oily Iodinated contrast media, and an implantable metal.

101. The composition of claim 98, further comprising at least one protein.

102. The composition of claim 85, wherein the composition further comprises at least one catalyst.

103. The composition of claim 102, wherein the at least one catalyst comprises tertiary amine, or an organometallic compound.

104. The composition of claim 85, further comprising surfactant.

105. The composition of claim 85, wherein the composition is adapted to stimulate bone growth when the composition contacts or is positioned in the vicinity of a bone of a mammal.

106. A composition comprising a polyester urethane and at least one filler material, wherein the composition is adapted to stimulate bone growth when the composition contacts or is positioned in the vicinity of a bone of a mammal.

107. The composition of claim 106, wherein the at least one filler material is present in an amount in the range of from about 0.01% to about 30% by weight.

108. The composition of claim 106, wherein the at least one filler material is selected from the group consisting of calcium carbonate, bone, calcium phosphate, calcium pyrophosphate, hydroxyapatite, poly methyl methacrylate, glass-ionomer, poly ether ether ketone, calcium sulfate, and tricalcium phosphate.

109. The composition of claim 108, wherein the at least one filler material is bone that is selected from the group consisting of demineralized bone, allograft bone, and autogenous bone.

110. The composition of claim 106, wherein the filler material is beta tricalcium phosphate.

111. The composition of claim 106 wherein the polyester urethane is present in an amount in the range of from about 70% to about 99.99% by weight.

112. The composition of claim 106 wherein the polyester urethane is a product of a process comprising the steps of:
forming a first compound by mixing a naturally occurring polyol with a biocompatible, synthetic polyol;
mixing the first compound with isocyanate; and
permitting the first compound and the isocyanate to react to form a polyester urethane.

113. The composition of claim 106 wherein the polyester urethane is a product of a process comprising the steps of:
mixing a naturally occurring polyol with an isocyanate;
permitting the naturally occurring polyol and the isocyanate to react to form a polyester urethane; and
permitting water to be present.

114. The composition of claim 106 wherein the polyester urethane is a product of a process comprising the steps of:
mixing a biocompatible, synthetic polyol with an isocyanate; and
permitting the biocompatible, synthetic polyol and the isocyanate to react to form a polyester urethane.

115. The composition of claim 106 wherein the polyester urethane is a product of a process comprising the steps of forming an isocyanate prepolymer by mixing a biocompatible, synthetic polyol with isocyanate;
mixing the isocyanate prepolymer with a naturally occurring polyol oil; and
permitting the isocyanate prepolymer and the naturally occurring polyol to react to form a polyester urethane.

116. The composition of claim 106 wherein the polyester urethane is a product of a process comprising the steps of:
forming an isocyanate prepolymer by mixing a biocompatible, synthetic polyol with isocyanate;
mixing the isocyanate prepolymer with a crosslinker or chain-extender; and
permitting the isocyanate prepolymer and the crosslinker or chain-extender to react to form a polyester urethane.

117. The composition of claim 106 having an average porosity in the range of from about 5 microns to about 500 microns.

118. The composition of claim 106, further comprising at least one substance selected from the group consisting of a radiotransparent substance and a radiopaque substance.

119. The composition of claim 118, wherein the at least one substance is at least one radiotransparent substance selected from the group consisting of gas, air, nitrogen gas, carbon dioxide, and oxygen gas.

120. The composition of claim 118, wherein the at least one substance is at least one radiopaque substance selected from the group consisting of insoluble zirconium oxide, a radioactive tracer, a Barium Sulfate contrast media, a gadolinium contrast media, a water-soluble Iodinated contrast media, an oily Iodinated contrast media, and an implantable metal.

121. The composition of claim 106, further comprising at least one protein.

122. The composition of claim 106, wherein the composition is in a moldable state at room temperature for at least about 20 minutes after formulation.

123. The composition of claim 122 wherein the composition begins to cure at about room temperature at a time in the range of from about 20 minutes to about 30 minutes after formulation.

124. The composition of claim 106 wherein the composition attains a final, cured state within about 48 hours after formulation.

125. The composition of claim 124 wherein the composition in its final, cured state has a compressive strength of at least about 50 MPa.

126. The composition of claim 124 wherein the composition in its final, cured state has a tensile strength of at least about 40 MPa.

127. The composition of claim 124 wherein the composition in its final, cured state has a Modulus of Elasticity of at least about 1,500 MPa.

128. The composition of claim 124, wherein the final, cured composition further comprises at least one substance selected from the group consisting of a radiotransparent substance and a radiopaque substance, and wherein the presence of the at least one substance does not substantially affect the mechanical properties of the final, cured composition.

129. The composition of claim 106, wherein the composition does not comprise any reactive isocyanate.

130. The composition of claim 106, wherein free powder is absent or substantially absent from the final, cured composition.

131. The composition of claim 106, wherein the mechanical properties of the composition are substantially unaffected when it is exposed to sterilization temperatures in an autoclave.

132. The composition of claim 106, wherein the composition is adhesive and cohesive.

133. The composition of claim 106, wherein the composition is bactericidal.

134. The composition of claim 106, wherein the composition is bacterial static.

135. A method of making a composition, comprising the steps of:
forming a first compound by mixing a naturally occurring polyol with a biocompatible, synthetic polyol; and
mixing the first compound with isocyanate.

136. The method of claim 135, further comprising the step of mixing at least one filler material with the first compound and the isocyanate, wherein the at least one filler material is selected from the group consisting of calcium carbonate, bone, calcium phosphate, calcium pyrophosphate, hydroxyapatite, poly methyl methacrylate, poly ether ether ketone, glass-ionomer, calcium sulfate, and tricalcium phosphate.

137. The method of claim 135, wherein the step of forming the first compound further comprises the step of mixing at least one catalyst with the naturally occurring polyol and the biocompatible, synthetic polyol.

138. The method of claim 137, wherein the at least one catalyst is a tertiary amine, or an organometallic compound.

139. The method of claim 135, further comprising the step of mixing surfactant with the first compound and the isocyanate.

140. The method of claim 135, further comprising the step of mixing at least one substance selected from the group consisting of at least one radiotransparent substance and at least one radiopaque substance with the first compound and the isocyanate.

141. The method of claim 135, further comprising the step of mixing at least one protein with the first compound and the isocyanate.

142. The method of claim 135, wherein the isocyanate is selected from the group consisting of an aromatic isocyanate, an aliphatic isocyanate, and a cycloaliphatic isocyanate.

143. A method of making a composition, comprising the steps of:
forming a first compound by mixing a naturally occuring polyol with a biocompatible, synthetic polyol;
mixing the first compound with isocyanate; and
permitting the first compound and the isocyanate to react to form a polyester urethane.

144. The method of claim 143, further comprising the step of mixing at least one filler material with the first compound and the isocyanate, wherein the at least one filler material is selected from the group consisting of calcium carbonate, bone, calcium phosphate, calcium pyrophosphate, hydroxyapatite, poly methyl methacrylate, poly ether ether ketone, glass-ionomer, calcium sulfate, and tricalcium phosphate.

145. The method of claim 143, wherein the step of forming the first compound further comprises the step of mixing at least one catalyst with the naturally occurring polyol and the biocompatible, synthetic polyol.

146. The method of claim 145, wherein the at least one catalyst is a tertiary amine.

147. The method of claim 143, further comprising the step of mixing surfactant with the first compound and the isocyanate.

148. The method of claim 143, further comprising the step of mixing at least one substance selected from the group consisting of at least one radiotransparent substance and at least one radiopaque substance with the first compound and the isocyanate.

149. The method of claim 143, further comprising the step of mixing at least one protein with the first compound and the isocyanate.

150. The method of claim 145, wherein the at least one filler material is present in the composition in an amount in the range of from about 0.01% to about 30% by weight.

151. The method of claim 143, wherein the isocyanate is selected from the group consisting of an aromatic isocyanate, an aliphatic isocyanate, and a cycloaliphatic isocyanate.

152. The method of claim 151, wherein the aromatic isocyanate is diphenylmethane diisocyanate.

153. The method of claim 143, wherein the isocyanate is a cycloaliphatic isocyanate.

154. The method of claim 143 wherein the naturally occurring polyol has at least one hydroxyl group.

155. The method of claim 143 wherein the naturally occurring polyol is selected from the group consisting of castor oil, safflower oil, lesquerella oil, a polyol obtained by chemical modification of a naturally occurring vegetable oil, a naturally occurring oil that has been trans-esterified, and a naturally occurring oil that has been hydrogenated.

156. The method of claim 143 wherein the biocompatible, synthetic polyol is selected from the group consisting of: a polycaprolactone polyol, a polyester polyol, a polyadipate polyol, and a polyol derived from a synthetic acid.

157. The method of claim 143, wherein the composition has a porosity, and further comprising the step of controlling the porosity of the composition.

158. The method of claim 157, wherein the composition comprises water, and wherein the step of controlling the porosity of the composition comprises the step of controlling the amount of water that is permitted to be present in the composition.

159. The method of claim 158, wherein the step of controlling the amount of water that is permitted to be present in the composition comprises injecting water into the composition.

160. The method of claim 158, wherein the water is present in the composition in an amount in the range of from about 0.1% to about 1% by weight.

161. The method of claim 158, wherein the step of controlling the porosity of the composition comprises a step selected from the group consisting of: injecting air into the composition, or permitting air to become entrained within the composition.

162. The method of claim 157, wherein the average porosity in the composition is in the range of from about 5 microns to about 500 microns.

163. The method of claim 143, wherein both the first compound and the isocyanate are liquids at room temperature.

164. The method of claim 143, further comprising the step of applying heat while mixing the first compound with isocyanate.

165. The method of claim 143, further comprising the step of removing heat while mixing the first compound with isocyanate.

166. The method of claim 143, wherein the composition is adhesive and cohesive.

167. The method of claim 143, wherein the composition is bactericidal.

168. The method of claim 143, wherein the composition is bacterial static.

169. The method of claim 143, wherein the composition is in a moldable state at room temperature for at least about 20 minutes after formulation.

170. The method of claim 143, further comprising the step of permitting the composition to attain a final, cured state.

171. The method of claim 170, wherein the composition further comprises at least one substance selected from the group consisting of a radiotransparent substance and a radiopaque substance, and wherein the presence of the at least one substance does not substantially affect the mechanical properties of the composition.

172. The method of claim 170, wherein free powder is absent or substantially absent from the composition.

173. The method of claim 170, wherein the composition does not comprise any reactive isocyanate.

174. The method of claim 170, wherein the mechanical properties of the composition are substantially unaffected when it is sterilized in an autoclave.

175. A method of making a composition, comprising the steps of:
mixing a naturally occurring polyol with isocyanate; and
permitting water to be present in the composition.

176. The method of claim 175, further comprising the step of mixing at least one filler material with the naturally occurring polyol and the isocyanate, wherein the at least one filler material is selected from the group consisting of calcium carbonate, bone, calcium phosphate, calcium pyrophosphate, hydroxyapatite, poly methyl methacrylate, poly ether ether ketone, glass-ionomer, calcium sulfate, and tricalcium phosphate.

177. The method of claim 175, further comprising the step of mixing at least one catalyst with the naturally occurring polyol and the isocyanate.

178. The method of claim 177, wherein the at least one catalyst is a tertiary amine, or an organometallic compound.

179. The method of claim 175, further comprising the step of mixing surfactant with the naturally occurring polyol and the isocyanate.

180. The method of claim 175, further comprising the step of mixing at least one substance selected from the group consisting of at least one radiotransparent substance and at least one radiopaque substance with the naturally occurring polyol and the isocyanate.

181. The method of claim 175, further comprising the step of mixing at least one protein with the naturally occurring polyol and the isocyanate.

182. The method of claim 175, wherein the isocyanate is selected from the group consisting of an aromatic isocyanate, an aliphatic isocyanate, and a cycloaliphatic isocyanate.

183. A method of making a composition, comprising the steps of:
mixing a naturally occurring polyol with isocyanate;
permitting the naturally occurring polyol and the isocyanate to react to form a polyester urethane; and
permitting water to be present in the composition.

184. The method of claim 183, further comprising the step of mixing at least one filler material with the naturally occurring polyol and the isocyanate, wherein the at least one filler material is selected from the group consisting of calcium carbonate, bone, calcium phosphate, calcium pyrophosphate, hydroxyapatite, poly methyl methacrylate, poly ether ether ketone, glass-ionomer, calcium sulfate, and tricalcium phosphate.

185. The method of claim 183, further comprising the step of mixing at least one catalyst with the naturally occurring polyol and the isocyanate.

186. The method of claim 185, wherein the at least one catalyst is a tertiary amine.

187. The method of claim 183, further comprising the step of mixing surfactant with the naturally occurring polyol and the isocyanate.

188. The method of claim 183, further comprising the step of mixing at least one substance selected from the group consisting of at least one radiotransparent substance and at least one radiopaque substance with the first compound and the isocyanate.

189. The method of claim 183, further comprising the step of mixing at least one protein with the naturally occurring polyol and the isocyanate.

190. The method of claim 185, wherein the at least one filler material is present in the composition in an amount in the range of from about 0.01% to about 30% by weight.

191. The method of claim 183, wherein the isocyanate is selected from the group consisting of an aromatic isocyanate, an aliphatic isocyanate, and a cycloaliphatic isocyanate.

192. The method of claim 191, wherein the aromatic isocyanate is diphenylmethane diisocyanate.

193. The method of claim 183, wherein the isocyanate is a cycloaliphatic isocyanate.

194. The method of claim 183 wherein the naturally occurring polyol has at least one hydroxyl group.

195. The method of claim 183 wherein the naturally occurring polyol is selected from the group consisting of castor oil, safflower oil, lesquerella oil, a polyol obtained by chemical modification of a naturally occurring vegetable oil, a naturally occurring oil that has been trans-esterified, and a naturally occurring oil that has been hydrogenated.

196. The method of claim 183, wherein the composition has a porosity, and further comprising the step of controlling the porosity of the composition.

197. The method of claim 196 wherein the step of controlling the porosity of the composition comprises the step of controlling the amount of water that is permitted to be present in the composition.

198. The method of claim 197, wherein the step of controlling the amount of water that is permitted to be present in the composition comprises injecting water into the composition.

199. The method of claim 197, wherein the water is present in the composition in an amount in the range of from about 0.1% to about 1% by weight.

200. The method of claim 196, wherein the step of controlling the porosity of the composition comprises a step selected from the group consisting of: injecting air into the composition, or permitting air to become entrained within the composition.

201. The method of claim 196, wherein the average porosity in the composition is in the range of from about 5 microns to about 500 microns.

202. The method of claim 183, wherein both the naturally occurring polyol and the isocyanate are liquids at room temperature.

203. The method of claim 183, further comprising the step of applying heat while mixing the naturally occurring polyol with isocyanate.

204. The method of claim 183, further comprising the step of removing heat while mixing the naturally occurring polyol with isocyanate.

205. The method of claim 183, wherein the composition is adhesive and cohesive.

206. The method of claim 183, wherein the composition is bactericidal.

207. The method of claim 183, wherein the composition is bacterial static.

208. The method of claim 183, wherein the composition is in a moldable state at room temperature for at least about 20 minutes after formulation.

209. The method of claim 183, further comprising the step of permitting the composition to attain a final, cured state.

210. The method of claim 209, wherein the composition further comprises at least one substance selected from the group consisting of a radiotransparent substance and a radiopaque substance, and wherein the presence of the at least one substance does not substantially affect the mechanical properties of the composition.

211. The method of claim 209, wherein free powder is absent or substantially absent from the composition.

212. The method of claim 209, wherein the composition does not comprise any reactive isocyanate.

213. The method of claim 209, wherein the mechanical properties of the composition are substantially unaffected when it is sterilized in an autoclave.

214. A method of making a composition, comprising the step of mixing a biocompatible, synthetic polyol with an isocyanate.

215. The method of claim 214, further comprising the step of mixing at least one filler material with the biocompatible, synthetic polyol and the isocyanate, wherein the at least one filler material is selected from the group consisting of calcium carbonate, bone, calcium phosphate, calcium pyrophosphate, hydroxyapatite, poly ether ether ketone, poly methyl Methacrylate, glass-ionomer, calcium sulfate, and tricalcium phosphate.

216. The method of claim 215 wherein the at least one filler material is present in the composition in an amount in the range of from about 0.01% to about 30% by weight.

217. The method of claim 214, further comprising the step of mixing surfactant with the biocompatible, synthetic polyol and the isocyanate.

218. The method of claim 214, further comprising the step of mixing at least one substance selected from the group consisting of at least one radiotransparent substance and at least one radiopaque substance with the biocompatible, synthetic polyol and the isocyanate.

219. The method of claim 214, further comprising the step of mixing at least one protein with the biocompatible, synthetic polyol and the isocyanate.

220. The method of claim 214, wherein the isocyanate is selected from the group consisting of an aromatic isocyanate, an aliphatic isocyanate, and a cycloaliphatic isocyanate.

221. A method of making a composition, comprising the steps of:
mixing a biocompatible, synthetic polyol with an isocyanate; and
permitting the biocompatible, synthetic polyol and the isocyanate to react to form a polyester urethane.

222. The method of claim 221, further comprising the step of mixing at least one filler material with the biocompatible, synthetic polyol and the isocyanate, wherein the at least one filler material is selected from the group consisting of calcium carbonate, bone, calcium phosphate, calcium pyrophosphate, hydroxyapatite, poly ether ether ketone, poly methyl methacrylate, glass-ionomer, calcium sulfate, and tricalcium phosphate.

223. The method of claim 222 wherein the at least one filler material is present in the composition in an amount in the range of from about 0.01% to about 30% by weight.

224. The method of claim 221, further comprising the step of mixing surfactant with the biocompatible, synthetic polyol and the isocyanate.

225. The method of claim 221, further comprising the step of mixing at least one substance selected from the group consisting of at least one radiotransparent substance and at least one radiopaque substance with the biocompatible, synthetic polyol and the isocyanate.

226. The method of claim 221, further comprising the step of mixing at least one protein with the biocompatible, synthetic polyol and the isocyanate.

227. The method of claim 221, wherein the isocyanate is selected from the group consisting of an aromatic isocyanate, an aliphatic isocyanate, and a cycloaliphatic isocyanate.

228. The method of claim 227, wherein the aromatic isocyanate is diphenylmethane diisocyanate.

229. The method of claim 221, wherein the isocyanate is a cycloaliphatic isocyanate.

230. The method of claim 221, wherein the composition has a porosity, and further comprising the step of controlling the porosity of the composition.

231. The method of claim 230, wherein the composition comprises water, and wherein the step of controlling the porosity of the composition comprises the step of controlling the amount of water that is permitted to be present in the composition.

232. The method of claim 231, wherein the step of controlling the amount of water that is permitted to be present in the composition comprises injecting water into the composition.

233. The method of claim 230, wherein the water is present in the composition in an amount in the range of from about 0.1% to about 1% by weight.

234. The method of claim 230, wherein the step of controlling the porosity of the composition comprises a step selected from the group consisting of: injecting air into the composition, or permitting air to become entrained within the composition.

235. The method of claim 230, wherein the average porosity in the composition is in the range of from about 5 microns to about 500 microns.

236. The method of claim 221, wherein both the biocompatible, synthetic polyol and the isocyanate are liquids at room temperature.

237. The method of claim 221, further comprising the step of applying heat while mixing the biocompatible, synthetic polyol with isocyanate.

238. The method of claim 221, further comprising the step of removing heat while mixing the biocompatible, synthetic polyol with isocyanate.

239. The method of claim 221, wherein the composition is adhesive and cohesive.

240. The method of claim 221, wherein the composition is bactericidal.

241. The method of claim 221, wherein the composition is bacterial static.

242. The method of claim 221, wherein the composition is in a moldable state at room temperature for at least about 20 minutes after formulation.

243. The method of claim 221, further comprising the step of permitting the composition to attain a final, cured state.

244. The method of claim 243, wherein the composition further comprises at least one substance selected from the group consisting of a radiotransparent substance and a radiopaque substance, and wherein the presence of the at least one substance does not substantially affect the mechanical properties of the composition.

245. The method of claim 243, wherein free powder is absent or substantially absent from the composition.

246. The method of claim 243, wherein the composition does not comprise any reactive isocyanate.

247. The method of claim 243, wherein the mechanical properties of the composition are substantially unaffected when it is sterilized in an autoclave.

248. A method of making a composition, comprising the steps of:
forming an isocyanate prepolymer by mixing a biocompatible, synthetic polyol with isocyanate; and
mixing the isocyanate prepolymer with a naturally occurring polyol.

249. The method of claim 248, further comprising adding at least one filler material, wherein the at least one filler material is selected from the group consisting of calcium carbonate, bone, calcium phosphate, calcium pyrophosphate, hydroxyapatite, poly ether ether ketone, poly methyl methacrylate, glass-ionomer, calcium sulfate, and tricalcium phosphate.

250. The method of claim 249 wherein the at least one filler material is present in the composition in an amount in the range of from about 0.01% to about 30% by weight.

251. The method of claim 248, wherein the step of forming the isocyanate prepolymer further comprises the step of mixing at least one catalyst with the isocyanate and the biocompatible, synthetic polyol.

252. The method of claim 251, wherein the at least one catalyst comprises a tertiary amine, or an organometallic compound.

253. The method of claim 248, further comprising the step of mixing surfactant with the isocyanate prepolymer, the naturally occurring polyol, and the at least one filler material.

254. The method of claim 248, further comprising the step of mixing at least one substance selected from the group consisting of at least one radiotransparent substance and at least one radiopaque substance with the isocyanate prepolymer, the naturally occurring polyol, and the at least one filler material.

255. The method of claim 248, further comprising the step of mixing at least one protein with the isocyanate prepolymer, the naturally occurring polyol, and the at least one filler material.

256. The method of claim 248, wherein the isocyanate is selected from the group consisting of an aromatic isocyanate, an aliphatic isocyanate, and a cycloaliphatic isocyanate.

257. A method of making a composition, comprising the steps of:
forming an isocyanate prepolymer by mixing a biocompatible, synthetic polyol with isocyanate;
mixing the isocyanate prepolymer with a naturally occurring polyol; and
permitting the isocyanate prepolymer and the naturally occurring polyol to react to form a polyester urethane.

258. The method of claim 257, further comprising adding at least one filler material, wherein the at least one filler material is selected from the group consisting of calcium carbonate, bone, calcium phosphate, calcium pyrophosphate, hydroxyapatite, poly ether ether ketone, poly methyl methacrylate, glass-ionomer, calcium sulfate, and tricalcium phosphate.

259. The method of claim 258 wherein the at least one filler material is present in the composition in an amount in the range of from about 0.01% to about 30% by weight.

260. The method of claim 257, wherein the step of forming the isocyanate prepolymer further comprises the step of mixing at least one catalyst with the isocyanate and the biocompatible, synthetic polyol.

261. The method of claim 260, wherein the at least one catalyst comprises a tertiary amine, or an organometallic compound.

262. The method of claim 258, further comprising the step of mixing surfactant with the isocyanate prepolymer, the naturally occurring polyol, and the at least one filler material.

263. The method of claim 258, further comprising the step of mixing at least one substance selected from the group consisting of at least one radiotransparent substance and at least one radiopaque substance with the isocyanate prepolymer, the naturally occurring polyol, and the at least one filler material.

264. The method of claim 258, further comprising the step of mixing at least one protein with the isocyanate prepolymer, the naturally occurring polyol, and the at least one filler material.

265. The method of claim 257, wherein the isocyanate is selected from the group consisting of an aromatic isocyanate, an aliphatic isocyanate, and a cycloaliphatic isocyanate.

266. The method of claim 265, wherein the aromatic isocyanate is diphenylmethane diisocyanate.

267. The method of claim 257 wherein the isocyanate is a cycloaliphatic isocyanate.

268. The method of claim 257, wherein the composition has a porosity, and further comprising the step of controlling the porosity of the composition.

269. The method of claim 268, wherein the composition comprises water, and wherein the step of controlling the porosity of the composition comprises the step of controlling the amount of water that is permitted to be present in the composition.

270. The method of claim 269, wherein the step of controlling the amount of water that is permitted to be present in the composition comprises injecting water into the composition.

271. The method of claim 269, wherein the water is present in the composition in an amount in the range of from about 0.1% to about 1% by weight.

272. The method of claim 268, wherein the step of controlling the porosity of the composition comprises a step selected from the group consisting of: injecting air into the composition, or permitting air to become entrained within the composition.

273. The method of claim 268, wherein the average porosity in the composition is in the range of from about 5 microns to about 500 microns.

274. The method of claim 257, wherein the isocyanate prepolymer and the naturally occurring polyol are liquids at room temperature.

275. The method of claim 257, further comprising the step of applying heat while mixing the isocyanate prepolymer, the naturally occurring polyol, and the at least one filler material.

276. The method of claim 257, further comprising the step of removing heat while mixing the isocyanate prepolymer, the naturally occurring polyol, and the at least one filler material.

277. The method of claim 257, wherein the composition is adhesive and cohesive.

278. The method of claim 257, wherein the composition is bactericidal.

279. The method of claim 257, wherein the composition is bacterial static.

280. The method of claim 257, wherein the composition is in a moldable state at room temperature for at least about 20 minutes after formulation.

281. The method of claim 257, further comprising the step of permitting the composition to attain a final, cured state.

282. The method of claim 281, wherein the composition further comprises at least one substance selected from the group consisting of a radiotransparent substance and a radiopaque substance, and wherein the presence of the at least one substance does not substantially affect the mechanical properties of the composition.

283. The method of claim 281, wherein free powder is absent or substantially absent from the composition.

284. The method of claim 281, wherein the composition does not comprise any reactive isocyanate.

285. The method of claim 281, wherein the mechanical properties of the composition are substantially unaffected when it is sterilized in an autoclave.

286. A method of making a composition, comprising the steps of:
forming an isocyanate prepolymer by mixing isocyanate with a biocompatible, synthetic polyol; and
mixing the isocyanate prepolymer with a crosslinker or chain-extender.

287. The method of claim 286, further comprising the step of mixing at least one filler material with the isocyanate prepolymer and the crosslinker or chain-extender, wherein at least one filler material is selected from the group consisting of calcium carbonate, bone, calcium phosphate, calcium pyrophosphate, hydroxyapatite, poly ether ether ketone, poly methyl methacrylate, glass-ionomer, calcium sulfate, and tricalcium phosphate.

288. The method of claim 287, wherein the at least one filler material is present in the composition in an amount in the range of from about 0.01% to about 30% by weight.

289. The method of claim 286, wherein the step of forming the isocyanate prepolymer further comprises the step of mixing at least one catalyst with the isocyanate and the biocompatible, synthetic polyol.

290. The method of claim 289, wherein the at least one catalyst is a tertiary amine, or an organometallic compound.

291. The method of claim 286, further comprising the step of mixing surfactant with the isocyanate prepolymer and the chain-extender or crosslinker.

292. The method of claim 286, further comprising the step of mixing at least one substance selected from the group consisting of at least one radiotransparent substance and at least one radiopaque substance with the isocyanate prepolymer and the chain-extender or crosslinker.

293. The method of claim 286, further comprising the step of mixing at least one protein with the isocyanate prepolymer and the chain extender or crosslinker.

294. The method of claim 286, wherein the isocyanate is selected from the group consisting of an aromatic isocyanate, an aliphatic isocyanate, and a cycloaliphatic isocyanate.

295. The method of claim 286 wherein the chain extender is selected from the group consisting of 1,4-butanediol, diethylene glycol, and 1,6 hexanediol.

296. The method of claim 286 wherein the crosslinker is selected from the group consisting of glycerine, trimethylolpropane, a trifunctional castor oil-based polyol, and a quadri-functional castor oil-based polyol.

297. A method of making a composition, comprising the steps of:
forming an isocyanate prepolymer by mixing isocyanate with a biocompatible, synthetic polyol;
mixing the isocyanate prepolymer with a crosslinker or chain-extender; and
permitting the isocyanate prepolymer and the crosslinker or chain-extender to react to form a polyester urethane.

298. The method of claim 297, further comprising the step of mixing at least one filler material with the isocyanate prepolymer and the crosslinker or chain-extender, wherein the at least one filler material is selected from the group consisting of calcium carbonate, bone, calcium phosphate, calcium pyrophosphate, hydroxyapatite, poly ether ether ketone, poly methyl methacrylate, glass-ionomer, calcium sulfate, and tricalcium phosphate.

299. The method of claim 298, wherein the at least one filler material is present in the composition in an amount in the range of from about 0.01% to about 30% by weight.

300. The method of claim 297, wherein the step of forming the isocyanate prepolymer further comprises the step of mixing at least one catalyst with the isocyanate and the biocompatible, synthetic polyol.

301. The method of claim 300, wherein the at least one catalyst is a tertiary amine, or an organometallic compound.

302. The method of claim 297, further comprising the step of mixing surfactant with the isocyanate prepolymer and the crosslinker or chain-extender.

303. The method of claim 297, further comprising the step of mixing at least one substance selected from the group consisting of at least one radiotransparent substance and at least one radiopaque substance with the isocyanate prepolymer and the crosslinker or chain-extender.

304. The method of claim 297, further comprising the step of mixing at least one protein with the isocyanate prepolymer and the crosslinker or chain-extender.

305. The method of claim 297, wherein the isocyanate is selected from the group consisting of an aromatic isocyanate, an aliphatic isocyanate, and a cycloaliphatic isocyanate.

306. The method of claim 305, wherein the aromatic isocyanate is diphenylmethane diisocyanate.

307. The method of claim 297 wherein the isocyanate is a cycloaliphatic isocyanate.

308. The method of claim 297, wherein the composition has a porosity, further comprising the step of controlling the porosity of the composition.

309. The method of claim 308, wherein the composition further comprises water, and wherein the step of controlling the porosity of the composition comprises the step of controlling the amount of water that is permitted to be present in the composition.

310. The method of claim 309, wherein the step of controlling the amount of water that is permitted to be present in the composition comprises injecting water into the composition.

311. The method of claim 309, wherein the water is present in the composition in an amount in the range of from about 0.1% to about 1% by weight.

312. The method of claim 308, wherein the step of controlling the porosity of the composition comprises a step selected from the group consisting of: injecting air into the composition, or permitting air to become entrained within the composition.

313. The method of claim 308, wherein the average porosity in the composition is in the range of from about 5 microns to about 500 microns.

314. The method of claim 297, wherein both the isocyanate prepolymer and the biocompatible, synthetic polyol are liquids at room temperature.

315. The method of claim 297, further comprising the step of applying heat while mixing the isocyanate with the biocompatible, synthetic polyol.

316. The method of claim 297, further comprising the step of removing heat while mixing the isocyanate with the biocompatible, synthetic polyol.

317. The method of claim 297, wherein the composition is adhesive and cohesive.

318. The method of claim 297, wherein the composition is bactericidal.

319. The method of claim 297, wherein the composition is bacterial static.

320. The method of claim 297, wherein the composition is in a moldable state at room temperature for at least about 20 minutes after formulation.

321. The method of claim 297, further comprising the step of permitting the composition to attain a final, cured state.

322. The method of claim 321, wherein the composition further comprises at least one substance selected from the group consisting of a radiotransparent substance and a radiopaque substance, and wherein the presence of the at least one substance does not substantially affect the mechanical properties of the composition.

323. The method of claim 321, wherein free powder is absent or substantially absent from the composition.

324. The method of claim 321, wherein the composition does not comprise any reactive isocyanate.

325. The method of claim 321, wherein the mechanical properties of the composition are substantially unaffected when it is sterilized in an autoclave.

326. A method of performing a medical procedure, comprising at least one step selected from the group consisting of:
applying a particular composition to at least one portion of a bone of a mammal;
positioning the particular composition in the vicinity of the bone;
dispensing the particular composition into an opening formed within or through at least one portion of the bone; and
positioning the particular composition between a first bone portion of the mammal and a second bone portion of the mammal for fusing the first bone portion to the second bone portion, wherein the particular composition:
stimulates bone growth; and
is a product of a process that comprises the steps of:
forming a first compound by mixing a naturally occurring polyol with a biocompatible, synthetic polyol;
mixing the first compound with isocyanate; and
permitting the first compound and the isocyanate to react to form a polyester urethane.

327. The method of claim 326 wherein the particular material does not substantially increase in temperature after being placed in at least the vicinity of at least one portion of a bone of a mammal.

328. The method of claim 327 wherein the temperature of the particular material does not substantially increase above about 45° C. after being placed in at least the vicinity of at least one portion of a bone of a mammal.

329. The method of claim 326 wherein the isocyanate is a cycloaliphatic isocyanate.

330. The method of claim 326, wherein the particular composition does not comprise any reactive isocyanate.

331. A method of performing a medical procedure, comprising at least one step selected from the group consisting of:
applying a particular composition to at least one portion of a bone of a mammal;
positioning the particular composition in the vicinity of the bone;
dispensing the particular composition into an opening formed within or through at least one portion of the bone; and
positioning the particular composition between a first bone portion of the mammal and a second bone portion of the mammal for fusing the first bone portion to the second bone portion, wherein the particular composition:
stimulates bone growth; and
is a product of a process that comprises the steps of:
mixing a naturally occurring polyol with isocyanate;
permitting the naturally occurring polyol and the isocyanate to react to form a polyester urethane; and
permitting water to be present in the composition.

332. The method of claim 331 wherein the particular material does not substantially increase in temperature after being placed in at least the vicinity of at least one portion of a bone of a mammal.

333. The method of claim 332 wherein the temperature of the particular material does not substantially increase above about 45° C. after being placed in at least the vicinity of at least one portion of a bone of a mammal.

334. The method of claim 331 wherein the isocyanate is a cycloaliphatic isocyanate.

335. The method of claim 331, wherein the particular composition does not comprise any reactive isocyanate.

336. A method of performing a medical procedure, comprising at least one step selected from the group consisting of:
applying a particular composition to at least one portion of a bone of a mammal;
positioning the particular composition in the vicinity of the bone;
dispensing the particular composition into an opening formed within or through at least one portion of the bone; and
positioning the particular composition between a first bone portion of the mammal and a second bone portion of the mammal for fusing the first bone portion to the second bone portion, wherein the particular composition:
stimulates bone growth; and
is a product of a process that comprises the steps of:
mixing a biocompatible, synthetic polyol with an isocyanate; and
permitting the biocompatible, synthetic polyol and the isocyanate to react to form a polyester urethane.

337. The method of claim 336 wherein the particular material does not substantially increase in temperature after being placed in at least the vicinity of at least one portion of a bone of a mammal.

338. The method of claim 337 wherein the temperature of the particular material does not substantially increase above about 45° C. after being placed in at least the vicinity of at least one portion of a bone of a mammal.

339. The method of claim 336 wherein the isocyanate is a cycloaliphatic isocyanate.

340. The method of claim 336, wherein the particular composition does not comprise any reactive isocyanate.

341. A method of performing a medical procedure, comprising at least one step selected from the group consisting of:
applying a particular composition to at least one portion of a bone of a mammal;
positioning the particular composition in the vicinity of the bone;
dispensing the particular composition into an opening formed within or through at least one portion of the bone; and
positioning the particular composition between a first bone portion of the mammal and a second bone portion of the mammal for fusing the first bone portion to the second bone portion, wherein the particular composition:
stimulates bone growth; and
is a product of a process that comprises the steps of:
forming an isocyanate prepolymer by mixing a biocompatible, synthetic polyol with isocyanate;
mixing the isocyanate prepolymer with a naturally occurring polyol; and
permitting the naturally occurring polyol and the isocyanate prepolymer to react to form a polyester urethane.

342. The method of claim 341 wherein the particular material does not substantially increase in temperature after being placed in at least the vicinity of at least one portion of a bone of a mammal.

343. The method of claim 342 wherein the temperature of the particular material does not substantially increase above about 45° C. after being placed in at least the vicinity of at least one portion of a bone of a mammal.

344. The method of claim 341 wherein the isocyanate is a cycloaliphatic isocyanate.

345. The method of claim 341, wherein the particular composition does not comprise any reactive isocyanate.

346. A method of performing a medical procedure, comprising at least one step selected from the group consisting of:
applying a particular composition to at least one portion of a bone of a mammal;
positioning the particular composition in the vicinity of the bone;
dispensing the particular composition into an opening formed within or through at least one portion of the bone; and
positioning the particular composition between a first bone portion of the mammal and a second bone portion of the mammal for fusing the first bone portion to the second bone portion, wherein the particular composition:
stimulates bone growth; and
is a product of a process that comprises the steps of:
forming an isocyanate prepolymer by mixing isocyanate with a biocompatible, synthetic polyol;
mixing the isocyanate prepolymer with a crosslinker or chain-extender; and
permitting the isocyanate prepolymer and the crosslinker or chain-extender to react to form a polyester urethane.

347. The method of claim 346 wherein the particular material does not substantially increase in temperature after being placed in at least the vicinity of at least one portion of a bone of a mammal.

348. The method of claim 347 wherein the temperature of the particular material does not substantially increase above about 45° C. after being placed in at least the vicinity of at least one portion of a bone of a mammal.

349. The method of claim 346 wherein the isocyanate is a cycloaliphatic isocyanate.

350. The method of claim 346, wherein the solidified, particular composition does not comprise any reactive isocyanate.

351. A method of performing a medical procedure, comprising the steps of:
forming a mold;
dispensing a liquid, particular composition into the mold, wherein the particular composition solidifies within the mold;
removing the solidified, particular composition from the mold; and
positioning the solidified, particular composition on a bone of a mammal or within an opening formed through or in the bone, wherein:
the positioned, particular composition stimulates bone growth; and
the particular composition is a product of a process that comprises the steps of:
forming a first compound by mixing a naturally occurring polyol with a biocompatible, synthetic polyol; and
mixing the first compound with isocyanate.

352. The method of claim 351 wherein the particular material does not substantially increase in temperature after being positioned on a bone of a mammal or within an opening formed through or in the bone.

353. The method of claim 352 wherein the temperature of the particular material does not substantially increase above about 45° C. after being positioned on a bone of a mammal or within an opening formed through or in the bone.

354. The method of claim 351 wherein the isocyanate is a cycloaliphatic isocyanate.

355. The method of claim 351, wherein the solidified, particular composition does not comprise any reactive isocyanate.

356. A method of performing a medical procedure, comprising the steps of:
forming a mold;
dispensing a liquid, particular composition into the mold, wherein the particular composition solidifies within the mold;
removing the solidified, particular composition from the mold; and
positioning the solidified, particular composition on a bone of a mammal or within an opening formed through or in the bone, wherein:
the positioned, particular composition stimulates bone growth; and
the particular composition is a product of a process that comprises the steps of:
mixing a naturally occurring polyol with isocyanate; and
permitting water to be present in the composition.

357. The method of claim 356 wherein the particular material does not substantially increase in temperature after being positioned on a bone of a mammal or within an opening formed through or in the bone.

358. The method of claim 357 wherein the temperature of the particular material does not substantially increase above about 45° C. after being positioned on a bone of a mammal or within an opening formed through or in the bone.

359. The method of claim 356 wherein the isocyanate is a cycloaliphatic isocyanate.

360. The method of claim 356, wherein the solidified, particular composition does not comprise any reactive isocyanate.

361. A method of performing a medical procedure, comprising the steps of:
forming a mold;
dispensing a liquid, particular composition into the mold, wherein the particular composition solidifies within the mold;
removing the solidified, particular composition from the mold; and
positioning the solidified, particular composition on a bone of a mammal or within an opening formed through or in the bone, wherein:
the positioned, particular composition stimulates bone growth;
and the particular composition is a product of a process that comprises the step of mixing a biocompatible, synthetic polyol with an isocyanate.

362. The method of claim 361 wherein the particular material does not substantially increase in temperature after being placed in at least the vicinity of at least one portion of a bone of a mammal.

363. The method of claim 362 wherein the temperature of the particular material does not substantially increase above about 45° C. after being placed in at least the vicinity of at least one portion of a bone of a mammal.

364. The method of claim 361 wherein the isocyanate is a cycloaliphatic isocyanate.

365. The method of claim 361, wherein the solidified, particular composition does not comprise any reactive isocyanate.

366. A method of performing a medical procedure, comprising the steps of:
forming a mold;
dispensing a liquid, particular composition into the mold, wherein the particular composition solidifies within the mold;
removing the solidified, particular composition from the mold; and
positioning the solidified, particular composition on a bone of a mammal or within an opening formed through or in the bone, wherein:
the positioned, particular composition stimulates bone growth; and
the particular composition is a product of a process that comprises the steps of:
forming an isocyanate prepolymer by mixing a biocompatible, synthetic polyol with isocyanate; and
mixing the isocyanate prepolymer with a naturally occurring polyol.

367. The method of claim 366 wherein the particular material does not substantially increase in temperature after being placed in at least the vicinity of at least one portion of a bone of a mammal.

368. The method of claim 367 wherein the temperature of the particular material does not substantially increase above about 45° C. after being placed in at least the vicinity of at least one portion of a bone of a mammal.

369. The method of claim 366 wherein the isocyanate is a cycloaliphatic isocyanate.

370. The method of claim 366, wherein the solidified, particular composition does not comprise any reactive isocyanate.

371. A method of performing a medical procedure, comprising the steps of:
forming a mold;
dispensing a liquid, particular composition into the mold, wherein the particular composition solidifies within the mold;
removing the solidified, particular composition from the mold; and
positioning the solidified, particular composition on a bone of a mammal or within an opening formed through or in the bone, wherein:
the positioned, particular composition stimulates bone growth; and
the particular composition is a product of a process that comprises the steps of:
forming an isocyanate prepolymer by mixing isocyanate with a biocompatible, synthetic polyol; and
mixing the isocyanate prepolymer with a crosslinker or chain-extender.

372. The method of claim 371 wherein the particular material does not substantially increase in temperature after being placed in at least the vicinity of at least one portion of a bone of a mammal.

373. The method of claim 372 wherein the temperature of the particular material does not substantially increase above about 45° C. after being placed in at least the vicinity of at least one portion of a bone of a mammal.

374. The method of claim 371 wherein the isocyanate is a cycloaliphatic isocyanate.

375. The method of claim 371, wherein the solidified, particular composition does not comprise any reactive isocyanate.

376. A kit for promoting bone growth, comprising:
a first container comprising a dispensing means and a first compound; and
a second container comprising a dispensing means and a second compound.

377. The kit of claim 376, wherein the first compound comprises a naturally occurring polyol.

378. The kit of claim 377 wherein the second compound comprises an isocyanate prepolymer comprising isocyanate.

379. The kit of claim 376 wherein the first compound comprises a biocompatible, synthetic polyol.

380. The kit of claim 379 wherein the second compound comprises isocyanate.

381. The kit of claim 376 wherein the first compound comprises a polyol comprising a naturally occurring polyol and a biocompatible, synthetic polyol.

382. The kit of claim 381 wherein the second compound comprises isocyanate.

383. The kit of claim 376, wherein the first container is a syringe.

384. The kit of claim 383, wherein the second container is a syringe.

385. The kit of claim 376 wherein the first container and the second container are packaged in a moisture resistant package.
